(19) United States
(12) Patent Application Publication
(10) Pub. No.: US 2010/0041736 A1
(43) Pub. Date: Feb. 18, 2010

(54) Isolated MCPIP and Methods of Use

(76) Inventors: Pappachan Kolattukudy, Orlando, FL (US); Jianli Niu, Orlando, FL (US); Asim Azfer, Orlando, FL (US)

Correspondence Address: Timothy H. Van Dyke, 390 No. Orange Avenue, Suite 2500, Orlando, FL, 32801, US

(21) Appl. No.: 12/539,907

(22) Filed: Aug. 12, 2009

Related U.S. Application Data
(63) Continuation of application No. 11/643,057, filed on Dec. 20, 2006, now abandoned.
(60) Provisional application No. 60/751,927, filed on Dec. 20, 2005.
(60) Provisional application No. 60/826,428, filed on Sep. 21, 2006.

(51) Int. Cl. A61K 31/7052; C12N 5/00; A61P 3/10; A61P 3/04

(52) U.S. Cl. 514/44 A; 435/375

(57) ABSTRACT

A monocyte chemoattractant protein (MCP-1)-inducible protein, MCPIP, its polynucleotide and amino acid sequences from mouse and human, and methods for its use are disclosed.

### CROSS-REFERENCE TO RELATED APPLICATIONS

This application is a continuation of U.S. Ser. No. 11/643,057 filed Dec. 20, 2006, which claims the benefit of priority of earlier-filed U.S. Provisional Patent Application No. 60/751,927 filed Dec. 20, 2005 and U.S. Provisional Patent Application No. 60/826,428 filed Sep. 21, 2006.

### STATEMENT OF GOVERNMENT RIGHTS

This invention was made with government grant support from the United States National Institutes of Health (HL69458 and K24-HL04208). The government has certain rights in this invention.

### FIELD OF THE INVENTION

The invention relates to cellular factors for modulating cellular pathways. More particularly, the invention relates to cellular transcription factors that affect inflammation and for methods for their therapeutic use.

### BACKGROUND OF THE INVENTION

Among the leading causes of mortality worldwide—cardiovascular disease, diabetes, stroke, cancer, and a variety of other diseases—many are, at least in part, caused by the body's own inflammatory response. Ischemic heart disease, for example, is the leading cause of death in the United States. Approximately 1.5 million people in the U.S. alone suffer heart attacks, a common complication of ischemic disease, and approximately ⅓ of those individuals experience a fatal attack. Ischemic disease has been associated with elevated markers of inflammation, and certain pro-inflammatory molecules are proposed to play a role in development of the disease state. Cancer has also been associated with inflammation, particularly chronic inflammation. According to the American Cancer Society, more than ten million people in the U.S. were living with cancer in 2002. Stroke can be a result of inflammation of the blood vessel walls. Stroke is the third leading cause of death in the United States and the most common cause of disability in adults. Each year more than 500,000 Americans experience a stroke, and about 150,000 die from stroke-related causes.

Molecules that contribute to the immune response have been associated with a variety of disease states. Among these molecules is, for example, monocyte chemoattractant protein (MCP-1, also known as CCL2). MCP-1 targets monocytes, T lymphocytes, and other cells expressing the C—C chemokine receptor (CCR2). MCP-1 is associated with monocyte recruitment, monocyte activation, and induction of the respiratory burst. MCP-1 has, however, also been shown to be elevated in ischemic heart disease, peripheral artery disease, atherosclerotic lesions, some types of tumors, tuberculosis, and sarcoidosis. MCP-1 has been proposed to contribute to progression of certain tumors, and treatment of immunodeficient mice bearing human breast carcinoma cells with a neutralizing antibody to MCP-1 resulted in significant increases in survival and inhibition of the growth of lung metastases.

Identifying molecules that explain the association between inflammation and cancer and cardiovascular disease provides an opportunity to develop therapeutic agents for the prevention and treatment of those diseases.

### SUMMARY OF THE INVENTION

The invention relates to an isolated monocyte chemoattractant protein inducible protein (MCPIP), an isolated nucleic acid encoding MCPIP, and an isolated amino acid sequence encoded by the nucleic acid. In one embodiment, the invention relates to an isolated human MCPIP nucleic acid or an isolated human MCPIP protein. In another embodiment, the invention relates to an isolated non-human mammalian (e.g., mouse) MCPIP nucleic acid or an isolated non-human mammalian MCPIP protein.

In one embodiment, the invention comprises an isolated nucleic acid encoding a polypeptide comprising an MCP-1 inducible cellular transcription factor. The nucleic acid may comprise a nucleotide sequence of SEQ ID NO:1 or SEQ ID NO:3 and the polypeptide may comprises at least about 10 residues of SEQ ID NO: 2 or SEQ ID NO: 4. In another aspect of the invention, the polypeptide may comprise at least about 20 amino acids of SEQ ID NO: 2 or SEQ ID NO: 4.

The invention also encompasses a substantially purified polypeptide, comprising an amino acid sequence selected from the group consisting of SEQ ID NO: 2, SEQ ID NO: 4, a variant of SEQ ID NO: 2 having at least 80% identity to SEQ ID NO: 2 which comprises a similar MCP-1-inducible transcription factor activity to that of a polypeptide comprising SEQ ID NO: 2; and a variant of SEQ ID NO: 4 having at least 80% identity to SEQ ID NO: 4 which comprises a similar MCP-1-inducible transcription factor activity to that of a polypeptide comprising SEQ ID NO: 4. A composition of the invention may also comprise a pharmaceutical carrier.

The invention also encompasses a catalytically active deletion mutant of a polypeptide comprising SEQ ID NO: 2 or SEQ ID NO: 4, wherein the deletion mutant lacks at least one amino acid of the polypeptide.

Also provided is a purified or isolated polynucleotide comprising a nucleic acid selected from the group consisting of SEQ ID NO:1 or SEQ ID NO: 3, a nucleic acid encoding the amino acid sequence of SEQ ID NO: 1 or SEQ ID NO: 3; and a nucleic acid which hybridizes with either nucleic acid under moderately stringent conditions and encodes a polypeptide having a similar MCP-1 inducible transcription factor activity to that of the polypeptide comprising SEQ ID NO: 2 or SEQ ID NO: 4.

The invention also relates to methods for treating diseases associated with elevated monocyte chemoattractant protein inducible protein (MCPIP), comprising inhibiting MCPIP expression, activation, nuclear localization or DNA binding. In another embodiment, the invention relates to methods for treating diseases wherein increasing cellular levels of MCPIP would provide a therapeutic benefit.

### BRIEF DESCRIPTION OF THE DRAWINGS

FIG. 1 A) Schematic representation of MCPIP showing putative domain structure of the human MCPP protein. B) Induction of human MCPP gene expression in human monocytes by treatment with 7 nM MCP-1 as detected by RT-PCR.

FIG. 2. Cell death caused by transfection of HEK293 cells with hMCPIP-GFP (stippled bar) or GFP alone (solid bar). Cells were transfected with MCPIP-GFP or GFP alone, harvested at either day 1 or day 5 after transfection and stained with TMR-red (TUNEL) (A) and with 0.4% Trypan Blue (B). More than 200 cells were examined for each time point and experiments were repeated 4 times.

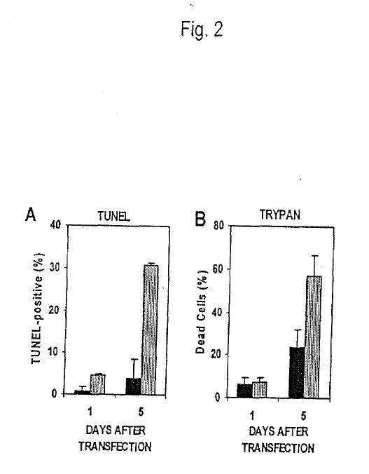

FIG. 3. In vitro transactivation of luciferase reporter gene by co-transfected hMCPIP or its mutants in cell cultures (A) and cell death induced by hMCPP or its mutants (B). Luciferase activity resulting from expression of pCGAL vector alone (vector), MCPIP-pCGAL fusion protein, or fusion proteins with mutants of MCPP, in HEK293 cell was measured. Cell death was assessed by trypan blue staining. NLS, mutated nuclear localization signal; PRO1, PR02, proline rich domain mutants 1 or 2; ZNF, zinc finger mutant.

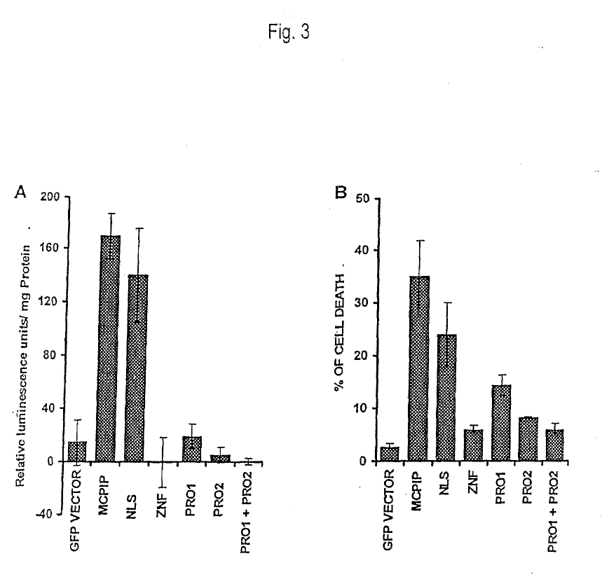

FIG. 4. Age-dependent increase in MCPIP gene expression in the hearts of wild type (stippled bar) and MCP mice (solid bar) (A) and compromise of left ventricular function with age as measured by reduction in fractional shortening (Baseline value was 50.7±4.2% measured by echocardiography). *p<0.05 (n=6, each group).

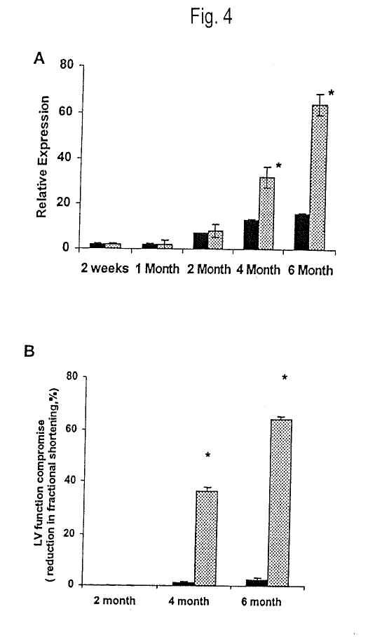

FIG. 5. In situ hydridization showing elevated expression of MCPIP in the cardiomyocytes of MCP-mice (A) and immunohistochemical detection of MCPIP in the hearts of MCP mice (B). Condensed nuclei staining with MCPIP was observed in cardiomyocytes and infiltrating inflammatory cells in MCP mice of 2 and 4 months of age (I and II), a strong staining for MCPIP was more prominent in the cardiomyocytes showing vacuolization (III, arrows) in 6 months old MCP mice with heart failure. (IV-VI, controls). (Original Magnification×400).

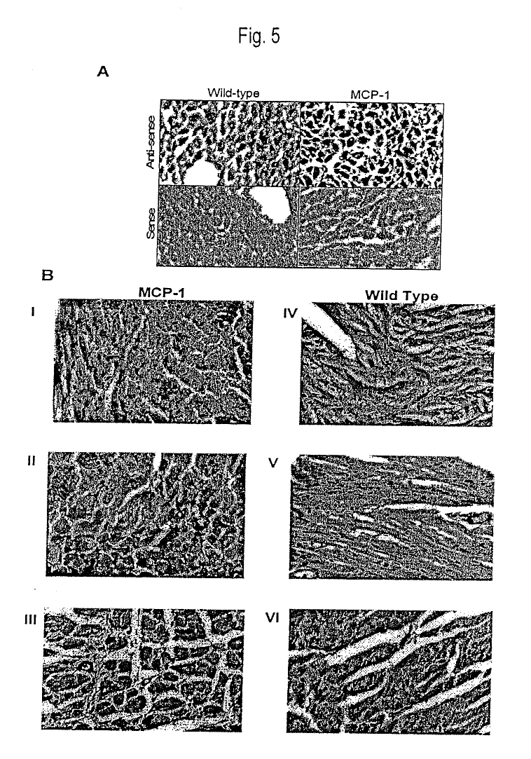

FIG. 6. Quantitative RT-PCR measurement of expression of CCR2 in the myocardium of MCP (stippled bar) and wild-type mice (solid bar) (A) and in situ hybridization showing expression of CCR2 in cardiomyocytes in 6 month-old of MCP-1-mice (B). *p<0.05 vs wild-type (n=6, each group).

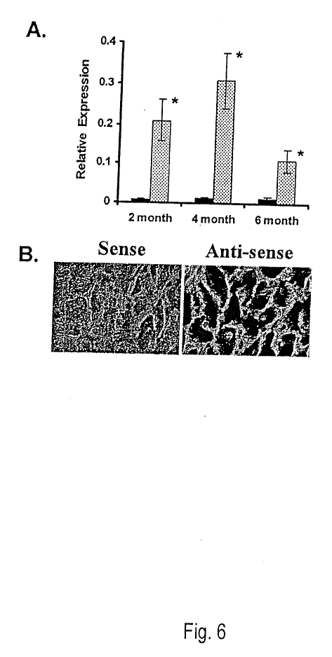

FIG. 7. MCPIP expression in individual ischemic and non-ischemic human heart tissues (A) and average values for MCPIP expression levels in ischemic and non-ischemic hearts (B). Age and sex of individuals are shown below each bar. *p<0.001 vs non-ischemic hearts.

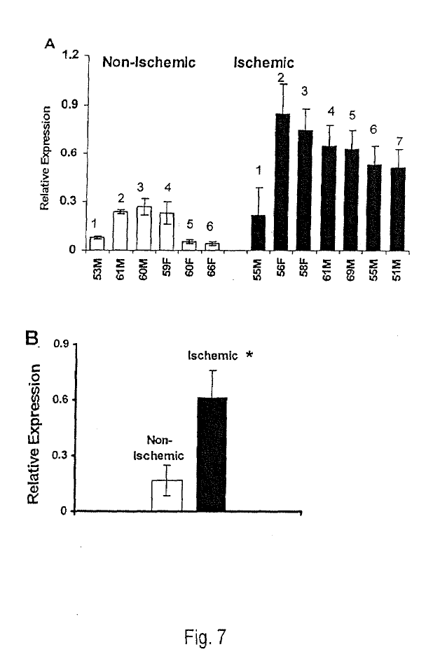

FIG. 8. Over-expression of hMCPIP induces HUVEC capillary-like tube formation. (A) Real-time PCR quantitative analysis of hMCPIP mRNA expression in HUVECs transfected with pEGFP/N1, pEGFP/hMCPIP, and pEGFP/hMCPIP plus hMCPIP-specific siRNA for 24 hours. *P<0.010 vs GFP control and hMCPIP-specific siRNA, n=3. (B) HUVECs were seeded on the surface of the polymerized ECMatrix™ (Millipore Corp.) for 24 hours after transfection. Phase-contrast photomicrographs (original magnification×100) were recorded on a digital camera. (C) Quantitative analysis of capillary-like tube formation of HUVECs. The mean percentage of branching over total cell clusters per field were calculated and expressed as a ratio to the control (untreated cells). *P<0.001vs control (untreated cells), GFP control and hMCPIP-specific siRNA, n=3.

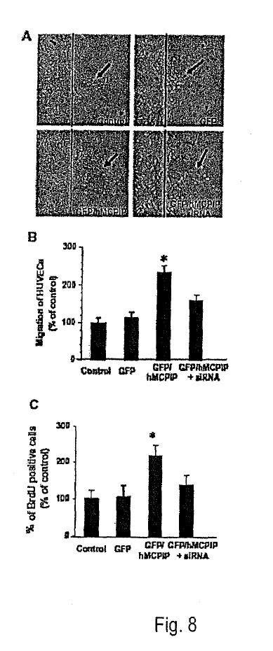

FIG. 9. Effects of hMCPIP on angiogenesis-related properties of HUVECs. (A) Phase-contrast photomicrographs of migration of HUVECs transfected with pEGFP/N1, pEGFP/hMCPIP, and pEGFP/hMCPIP plus hMCPIP-specific siRNA for 24 hours after wounding (original magnification×100). The wound margin and migrated cells were indicated with black outline and arrows, respectively. (B) Quantitation of HUVEC migration across the wound and results were expressed as a percentage of migration of control (untreated cells). *P<0.001 vs control (untreated cells), GFP control and hMCPIP-specific siRNA, n=3. (C, D) Cell proliferation was measured by BrdU incorporation and apoptosis was detected by DAPI nuclear staining in HUVECs transfected with pEGFP/N1, pEGFP/hMCPIP, and pEGFP/hMCP1P plus hMCPIP-specific siRNA for 24 hours, and expressed as a percentage of proliferation or apoptosis displayed by control (untreated cells). *P<0.001 vs control, GFP control and HMCPIP-specific siRNA, n=3.

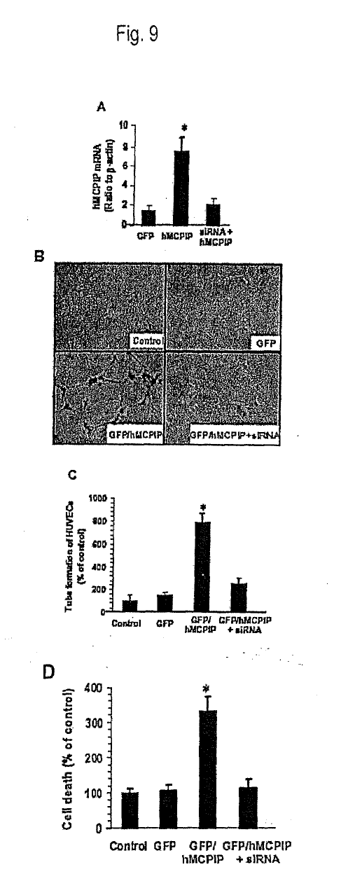

FIG. 10. hMCP1P transcriptionally activates the cadherin-12 and cadherin-19 promoters. HEK293 cells transfected with pEGFP/hMCPIP or pEGFP for 12 hours. Cells were cross-linked with formaldehyde and lysates were incubated with rabbit hMCPIP antibody for immunoprecipitation. The DNA purified from the precipitate was recovered, cloned in pCR-II-TOPO (Invitrogen, Carlsbad, Calif.) Blunt vector and plasmid DNAs were sequenced using SP6 promoter primer and T7 promoter primer. MCPIP bound to cadherin-12 and cadherin-19 promoters and these results were further confirmed by real-time PCR (B). Electrophoretic mobility shift analysis demonstrated binding specificity of hMCPIP selectively to genes encoding cadherin-12 (C) and cadherin-19 (D).

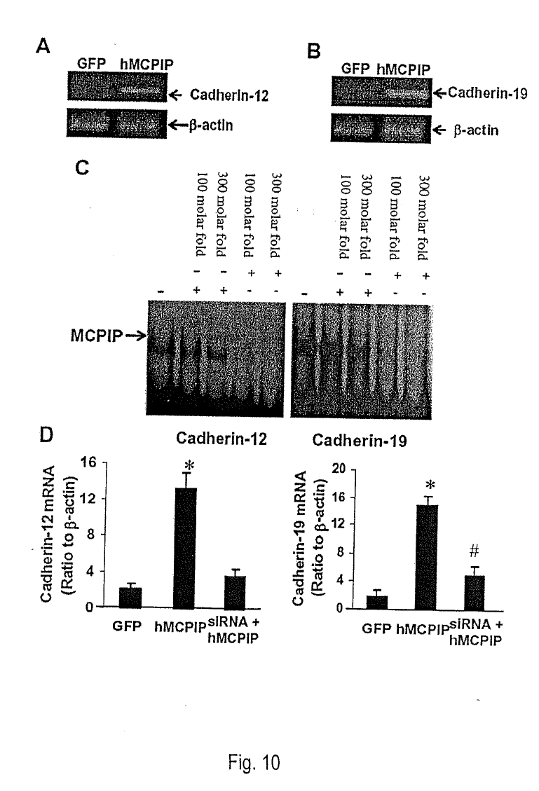

FIG. 11. siRNA-mediated knockdown of hMCPIP attenuates MCP-1-induced angiogenic activity. (A) HUVECs were treated with MCP-1 or with hMCPIP-specific siRNA for 24 hours, and hMCPIP mRNA expression was detected by RT-PCR. P-actin was amplified as internal control. (B) Quantitative analysis of hMCPIP expression in HUVECs treated with MCP-1 or hMCPIP siRNA by real-time PCR. *P<0.001 vs control (untreated cells) and hMCPIP-specific siRNA, n=3. (C) Phase-contrast photomicrographs of HUVECs treated with MCP-1 or MCP-1 plus hMCPIP-specific siRNA for 24 hours (original magnification×100).

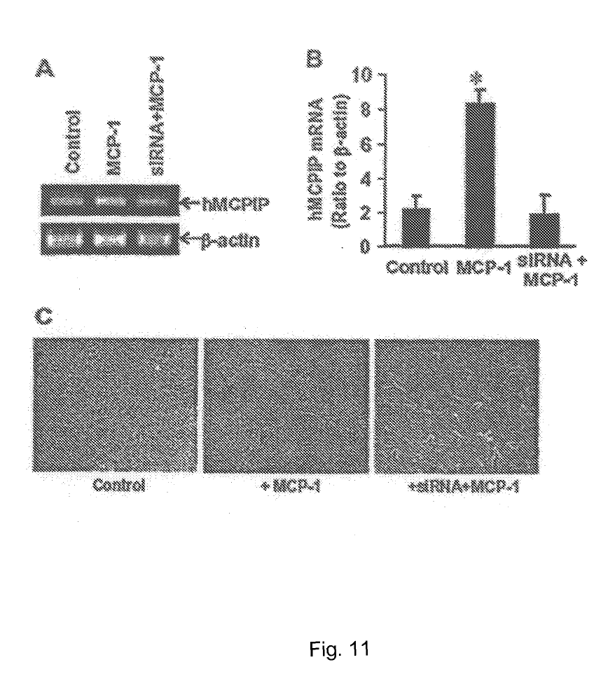

FIG. 12. Quantitative analysis of tube formation (A) of HUVECs treated with MCP-1 or MCP-1 plus hMCPIP-specific siRNA. *P<0.001 vs control and hMCPIP-specific siRNA; *P<0.05 vs control, n=3. HUVECs were treated with MCP-1 or with hMCPIP-specific siRNA for 24 hours, and mRNA expression of both cadherin-12 (B) and cadherin-19 (C) were detected by RT-PCR, respectively. β-actin was amplified as internal control. Quantitative analysis of cadherin-12 cadherin-19 mRNA expression in HUVECs treated with MCP-1 or hMCPIP siRNA by real-time PCR. *P<0.001 vs control and hMCPIP-specific siRNA, n=3.

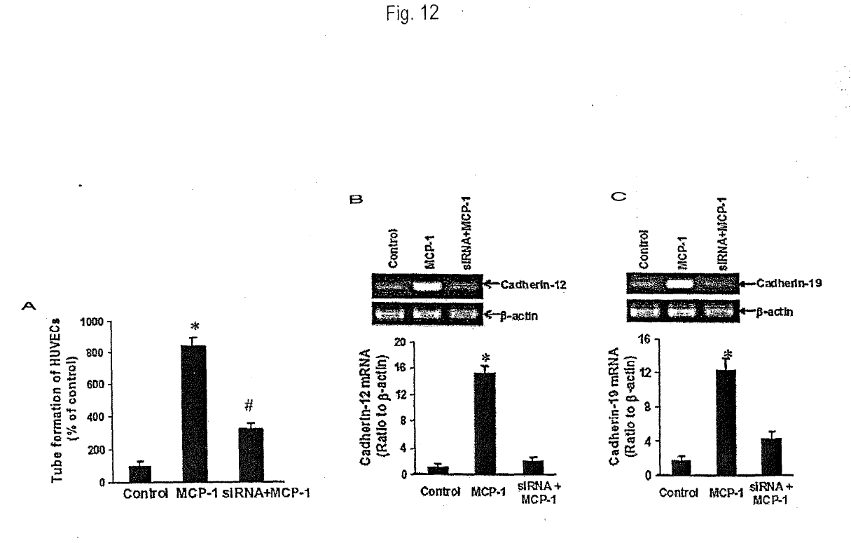

### DETAILED DESCRIPTION

The inventors have identified a novel transcription factor that they have designated MCPIP (MCP-1-induced protein), which they initially isolated from human monocytes after stimulation with MCP-1. The nucleotide (SEQ ID NO: 1) and amino acid (SEQ ID NO: 2) sequences of isolated human MCPIP were deposited with GenBank under accession number AY920403 and the nucleotide (SEQ ID NO: 3) and amino acid (SEQ ID NO: 4) sequences of isolated mouse MCPIP were deposited with GenBank under accession number AY920404.

MCPIP has been shown by the inventors to be induced by several stress factors that cause cell death. For example, hydrogen peroxide (H₂O₂), Staurosporine (STS), and Nitroprusside Sodium (NPS), agents known to cause cell death, induced MCPIP and MCP-1 synthesis in RAW cells (murine macrophage cell line), but both were inhibited by treatment of the cells with MCPIP siRNA. Treatment of cells with non-specific siRNA produced no such inhibitory effect.

MCPIP localizes to the nucleus, and mutation of the DNA-binding domain of MCPIP renders it non-functional. The inventors have demonstrated that MCPIP induces expression of ltv, creld2, ufm1 and lzp. They also demonstrated that MCP-1 induces expression of the same genes, and that induction of expression of the genes by MCP-1 can be inhibited by treatment of cells with siRNA specific for MCPIP. Non-specific siRNA had no such inhibitory effect. The inventors have also demonstrated that MCPIP induces expression of endothelial cell marker Flk1.

The inventors have also demonstrated that MCPIP expression correlates with the development of cardiac ischemia. They have shown that MCP-1 induces cell death in cardiomyoblast cell line H9C2 via activation of MCPIP. Adenoviral expression of MCPIP in H9C2 caused cell death, with a concomitant increase in production of reactive oxygen species (ROS). Treatment of cells with iNOS inhibitor (1400w) and NADH oxidase inhibitor (Tiron, 4,5-dihydroxy-m-benzenedisulfonic acid disodium salt) inhibited MCPIP-induced cell death.

Quantitative real-time polymerase chain reaction (qRTPCR) demonstrated that there is an increase in expression of mcpip and induction of a series of MCPIP-induced genes as high fat diet-fed mice develop mark weight gain, increased mass of white adipose tissue and increased fasting glucose levels.

Aspects of the invention therefore include polynucleotides encoding at least one mammalian MCPIP and amino acid sequences representing at least one MCPIP protein. Aspects of the invention also include subunits or variants of polynucleotides or MCPIP proteins or peptides encoded by those polynucleotides.

It is well known in the art that a single amino acid may be encoded by more than one nucleotide codon—and that the nucleotide sequence may be easily modified to produce an alternate nucleotide sequence that encodes the same peptide. Therefore, alternate embodiments of the present invention include alternate DNA sequences encoding peptides containing the amino acid sequences described for MCPIP. DNA sequences encoding peptides containing the claimed amino acid sequence include DNA sequences which encode any combination of the claimed sequence and any other amino acids located N-terminal or C-terminal to the claimed amino acid sequence.

It is to be understood that amino acid and nucleic acid sequences may include additional residues, particularly N- or C-terminal amino acids or 5′ or 3′ nucleotide sequences, and still be essentially as set forth in the sequences disclosed herein, as long as the sequence produces a functionally similar polypeptide or protein. A nucleic acid fragment of almost any length may be employed, and may be combined with other DNA sequences, such as promoters, polyadenylation signals, additional restriction enzyme sites, multiple cloning sites, other coding segments, and the like. Therefore, overall length may vary considerably.

MCPIP polypeptides, as used herein, may comprise short fragments of proteins often referred to as peptides, as well as longer fragments generally referred to as polypeptides, and full-length proteins. These polypeptides can be prepared by standard peptide synthesis methods known to those of skill in the art, but may also be produced using an expression vector having a polynucleotide sequence encoding the polypeptide(s) of choice operably linked to appropriate promoter, terminator, and other functional sequences (such as a sequence encoding a purification tag) to facilitate expression and purification of the peptides.

It is to be understood that amino acid and nucleic acid sequences may include additional residues, particularly N- or C-terminal amino acids or 5′ or 3′ nucleotide sequences, and still be essentially as set forth in the sequences disclosed herein, as long as the sequence confers MCP-1 inducible transcription factor activity upon the polypeptide or protein moiety of the expressed protein. Nucleic acids which hybridize with a nucleic acid encoding the amino acid sequence of SEQ ID NO: 2 or SEQ ID NO: 4 under stringent conditions and encode a polypeptide having a similar MCP-1 inducible transcription factor activity to that of a polypeptide comprising SEQ ID NO: 2 or SEQ ID NO: 4 are also included as embodiments of the present invention.

The term “moderately stringent conditions”, as used herein, means conditions in which non-specific hybridization will not generally occur. Hybridization under such conditions can be performed based on the description provided in Molecular Cloning: A Laboratory Manual 2nd ed., published by cold Spring Harbor Laboratory in 1989, edited by T. Maniatis et al. For example, stringent conditions include incubation with a probe in 6×SSC containing 0.5% SDS, 5×Denhardt's solution and 100 micrograms/ml salmon sperm DNA at 60° C.

Additional nucleic acid bases may be added either 5′ or 3′ to the MCPIP ORF, and may be combined with other DNA sequences, such as promoters, polyadenylation signals, additional restriction enzyme sites, multiple cloning sites, other coding segments, and the like. Therefore, overall length of such a polynucleotide may vary considerably. In a method described by the present invention, a nucleotide sequence of SEQ ID NO: 1 is inserted into a protein expression vector to produce a protein which can be used to synthesize a DNA copy of an RNA molecule. The DNA can then be amplified to form multiple copies.

“Control sequences” are those DNA sequences that are necessary for the expression of a protein from a polynucleotide sequence containing such a sequence, operably linked to the polynucleotide sequence encoding the protein. These sequences include prokaryotic sequences such as, for example, promoters, operators, and ribosome binding sites, and eukaryotic sequences such as, for example, promoters, enhancers, and polyadenylation signals. “Expression systems” are DNA sequences (such as, for example, plasmids) appropriate for expression of a target protein in a particular host cell, these sequences comprising appropriate control sequences for protein expression in the host cell operably linked to the polynucleotide sequence encoding the target protein.

It is to be understood that a “variant” of a polypeptide is not completely identical to the native protein. A variant MCPIP protein, for example, can be obtained by altering the amino acid sequence by insertion, deletion or substitution of one or more amino acids. The amino acid sequence of the protein can be modified, for example, by substitution to create a polypeptide having substantially the same or improved qualities as compared to the native polypeptide. The substitution may be a conserved substitution. A “conserved substitution” is a substitution of an amino acid with another amino acid having a side chain that is similar in polar/nonpolar nature, charge, or size. The 20 essential amino acids can be grouped as those having nonpolar side chains (alanine, valine, leucine, isoleucine, proline, phenylalanine, and tryptophan), uncharged polar side chains (methionine, glycine, serine, threonine, cystine, tyrosine, asparagine and glutamine), acidic side chains (aspartate and glutamate), and basic side chains (lysine, arginine, and histidine). Conserved substitutions might include, for example, Asp to Glu, Asn, or Gln; His to Lys, Arg or Phe; Asn to Gln, Asp or Glu; and Ser to Cys, Thr or Gly. Alanine, for example, is often used to make conserved substitutions.

To those of skill in the art, variant polypeptides can be obtained by substituting a first amino acid for a second amino acid at one or more positions in the polypeptide structure in order to affect biological activity. Amino acid substitutions may, for example, induce conformational changes in a polypeptide that result in increased biological activity.

Those of skill in the art may also make substitutions in the amino acid sequence based on the hydrophilicity index or hydropathic index of the amino acids. A variant amino acid molecule of the present invention, therefore, has less than one hundred percent, but at least about fifty percent, and preferably at least about eighty to about ninety percent amino acid sequence homology or identity to the amino acid sequence of a polypeptide comprising SEQ ID NO: 2, or a polypeptide encoded by SEQ ID NO: 4. Therefore, the amino acid sequence of the variant MCPIP protein corresponds essentially to the native MCPIP protein amino acid sequence. As used herein, “corresponds essentially to” refers to a polypeptide sequence that will elicit a similar biological and enzymatic activity to that generated by a MCPIP protein comprising SEQ ID NO 2 or SEQ ID NO: 4, such activity being at least about 70 percent that of the native MCPIP protein, and more preferably greater than 90 percent of the activity of the native MCPIP protein.

A variant of the MCPIP protein may include amino acid residues not present in a corresponding MCPIP protein comprising SEQ ID NO 2, or may include deletions relative to the MCPIP protein comprising SEQ ID NO 2. A variant may also be a truncated “fragment,” as compared to the corresponding protein comprising SEQ ID NO 2, the fragment being only a portion of the full-length protein.

Expression vectors may be chosen from among those readily available for prokaryotic or eukaryotic expression systems. Expression system vectors, which incorporate the necessary regulatory elements for protein expression, as well as restriction endonuclease sites that facilitate cloning of the desired sequences into the vector, are known to those of skill in the art. A number of these expression vectors are commercially available.

An expression vector host cell system can be chosen from among a number of such systems that are known to those of skill in the art. In one embodiment of the invention, the protein can be expressed in E. coli. In alternate embodiments of the present invention, the enzyme may be expressed and purified using other bacterial expression systems, viral expression systems, eukaryotic expression systems, or cell-free expression systems. Cellular hosts used by those of skill in the art for expression of various proteins include, but are not limited to, Bacillus subtilis, yeast such as Saccharomyces cerevisiae, Saccharomyces carlsbergenesis, Saccharomyces pombe, and Pichia pastoris, as well as mammalian cells such as 3T3, HeLa, and Vero. The expression vector chosen by one of skill in the art will include promoter elements and other regulatory elements appropriate for the host cell or cell-free system in which the recombinant DNA sequence encoding the enzyme will be expressed. In mammalian expression systems, for example, suitable expression vectors can include DNA plasmids, DNA viruses, and RNA viruses. In bacterial expression systems, suitable vectors can include plasmid DNA and bacteriophage vectors.

One group of vectors that can be used to express and facilitate purification of the protein include those vectors that encode the polyhistidine (6×His) sequence and an epitope tag to allow rapid purification of the fusion protein with a nickel-chelating resin, along with protein detection with specific antibodies to detect the presence of the secreted protein. An example of such a vector for expression in mammalian cells is the pcDNA3.1/V5-His-TOPO eukaryotic expression vector (Invitrogen). In this vector, the fusion protein can be expressed at high levels under the control of a strong cytomegalovirus (CMV) promoter. A C-terminal polyhistidine (6×His) tag enables fusion protein purification using nickel-chelating resin. Secreted protein produced by this vector can be detected using an anti-His (C-term) antibody.

Bacterial protein, bacterial expression systems may include, for example, the pMAL system (New England Biolabs, Beverly, Mass.) which utilizes a maltose binding protein fusion to facilitate purification, and the Impact-CN Protein Fusion and Purification System (New England Biolabs).

A baculovirus expression system can be used for production of a target protein such as the enzyme of the present invention. A commonly used baculovirus is AcMNPV. Cloning of the target protein DNA can be accomplished by using homologous recombination. The target protein DNA sequence is cloned into a transfer vector containing a baculovirus promoter flanked by baculovirus DNA, particularly DNA from the polyhedrin gene. This DNA is transfected into insect cells, where homologous recombination occurs to insert the target protein into the genome of the parent virus. Recombinants are identified by altered plaque morphology.

Proteins as described above can also be produced in the method of the present invention by mammalian viral expression systems. The Sindbis viral expression system, for example, can be used to express proteins at high levels. Sindbis vectors have been described, for example, in U.S. Pat. No. 5,091,309 (Schlesinger et al.), incorporated herein by reference. Sindbis expression vectors, such as pSinHis (Invitrogen, Carlsbad, Calif.) can be used to express the MCPIP protein under the direction of the subgenomic promoter PSG. In vitro transcribed RNA molecules encoding the fusion protein and the Sindbis proteins required for in vivo RNA amplification can be electroporated into baby hamster kidney (BHK) cells using methods known to those of skill in the art. Alternatively, the RNA encoding the MCPIP protein and Sindbis proteins required for in vivo RNA amplification can be cotransfected with helper RNA that permits the production of recombinant viral particles. Viral particles containing genetic material encoding the fusion protein can then be used to infect cells of a wide variety of cell types, including mammalian, avian, reptilian, and Drosophila. Fusion protein expressed from the pSinHis (Invitrogen) vector can be detected with antibody to an Anti-Xpress™ epitope encoded by the vector sequence. The pSinHis vector also includes a polyhistidine tag which provides a binding site for metal-chelating resins to facilitate purification of the expressed fusion protein. Furthermore, an enterokinase cleavage site located between the histidine tag and the fusion protein allows the histidine tag to be enzymatically removed following purification.

An ecdysone-inducible mammalian expression system (Invitrogen, Carlsbad, Calif.) can also be used to express a target protein. Vectors used in the ecdysone-inducible mammalian expression system can be organized to produce the target protein by expressing the target protein from the expression cassette. With the ecdysone-inducible system, higher levels of protein production can be achieved by use of the insect hormone 20-OH ecdysone to activate gene expression via the ecdysone receptor. An inducible expression plasmid provides a multiple cloning site, into which the nucleotide sequence of the MCPIP protein can be ligated. The expression vector contains ecdysone response elements upstream of the promoter (a minimal heat shock promoter) and the multiple cloning site. Co-transfection of a second plasmid, pVgRXR (Invitrogen), provides the receptor subunits to make the cell responsive to the steroid hormone ecdysone analog, ponasterone A. A control expression plasmid containing the lacZ gene can be cotransfected with pVgRXR to provide a marker for transfected cells. Upon induction with ponasterone A, the control plasmid expresses β-galactosidase. Co-transfection of the inducible expression construct and pVgRXR into the mammalian cell of choice can be accomplished by any of the standard means known to those of skill in the art. These include, for example, calcium phosphate transfection, lipid-mediated transfection, and electroporation. Levels of expression of the fusion protein in this system can be varied according to the concentration and length of exposure to ponasterone. Stable cell lines that constitutively express the MCPIP protein can be established using Zeocin.TM. (Invitrogen), a bleomycin/phleomycin-type antibiotic isolated from Streptomyces, and neomycin or hygromycin.

Yeast host cells, such as Pichia pastoris, can also be used for the production of the MCPIP protein. Expression of heterologous proteins from plasmids transformed into Pichia has previously been described by Sreekrishna, et al. (U.S. Pat. No. 5,002,876, incorporated herein by reference). Vectors for expression in Pichia of a MCPIP protein are commercially available as part of a Pichia Expression Kit (Invitrogen, Carlsbad, Calif.). Pichia pastoris is a methylotrophic yeast, which produces large amounts of alcohol oxidase to avoid the toxicity of hydrogen peroxide produced as a result of methanol metabolism. Alcohol oxidase gene expression is tightly regulated by the AOX1 and AOX2 promoters. In Pichia expression vectors, high levels of expression are produced under the control of these promoters. Ohi, et al. (U.S. Pat. No. 5,683,893, incorporated herein by reference) have previously described a mutant AOX2 promoter capable of producing enhanced expression levels.

Polypeptides of the invention may be delivered to a cell via attachment of one or more polypeptides to cell permeable, or “importation competent” signal peptide sequences, and membrane translocation sequences that have been shown to facilitate the transport of attached peptides and proteins into cells. Several sequences of this kind have previously been described, including the hydrophobic region of the signal sequence of Kaposi fibroblast growth factor which has been fused to the nuclear localization sequence (NLS) of p50 to produce the peptide known as SN50 (U.S. Pat. No. 5,807,746, Lin et al.). Polypeptides may also be delivered via a membrane translocating sequence described in U.S. Pat. Nos. 6,248,558; 6,432,680; and 6,780,843 (Rojas et al.). MCPIP, or a nuclear localization sequence that blocks nuclear localization of MCPIP, may also be delivered via the cell-permeable sequence described in United States Patent Application Number 20060099275 (Lin and Budu). Other membrane-translocating sequences are also well-known to those of skill in the art. Non-invasive delivery of proteins via membrane translocating peptides is discussed by Hawiger in Curr. Opin Chem. (1999) 3: 89-94, and multiple examples of both in vitro and in vivo use of membrane translocation via cell-permeable peptide sequences are available in the literature. The HIV-Tat peptide, for example, has been used in a number of studies to deliver cargo peptides to target cells (Ribeiro, M. M., et al. Biochem. Biophys. Res. Commun. (2003) 305(4): 876-81; Jung, H. J., et al. Biochem. Biophys. Res. Commun. (2006) 345(1): 222-228; Barnett, E. M., et al. Invest. Opthalmol. Vis. Sci. (2006) 47(6): 2589-2595; Hoque, M., et al. J. Biol. Chem. (2005) 280(14): 13648-13657; Mondal D., et al. Exp. Biol. Med. (2005) 230(9): 631-644; Kittiworakarn, J., et al. J. Biol. Chem. (2006) 281(6): 3105-3115).

Polynucleotides encoding all or a part of the amino acid sequence of MCPIP may be delivered in vitro or in vivo by a variety of means known to those of skill in the art, such as, for example, viral gene delivery, naked DNA, delivery via cationic lipid carriers, and plasmid DNA/polylysine complexes.

As used herein, MCPIP polypeptides include variants or biologically active fragments of the peptides, as well as peptides which may contain additional amino acids either N-terminal or C-terminal (or both) to the disclosed sequences, their derivatives, variants, or functional counterparts. A “functional counterpart” can include, for example, a peptide nucleic acid (PNA). A “variant” of the peptide is not completely identical to a disclosed MCPIP polypeptide sequence. A variant, given the disclosure of the present invention, can be obtained by altering the amino acid sequence by insertion, deletion or substitution of one or more amino acid. The amino acid sequence of a disclosed peptide can be modified, for example, by substitution to create a peptide having substantially the same or improved qualities. The substitution may be a conserved substitution. A “conserved substitution” is a substitution of an amino acid with another amino acid having a side chain that is similar in polar/nonpolar nature, charge, or size. The 20 essential amino acids can be grouped as those having nonpolar side chains (alanine, valine, leucine, isoleucine, proline, phenylalanine, and tryptophan), uncharged polar side chains (methionine; glycine, serine, threonine, cysteine, tyrosine, asparagine and glutamine), acidic side chains (aspartate and glutamate) and basic side chains (lysine, arginine, and histidine). Conserved substitutions might include, for example, Asp to Glu, Asn or Gln; His to Lys, Arg or Phe; Asn to Gln, Asp or Glu, Leu to Ile or Val, and Ser to Cys, Thr or Gly. Alanine is commonly used to make conserved substitutions.

To those of skill in the art, variant polypeptides can be obtained by substituting a first amino acid for a second amino acid at one or more positions in the peptide structure in order to affect biological activity. Amino acid substitutions may, for example, induce conformational changes in a polypeptide that result in increased biological activity. Those of skill in the art may also make substitutions in the amino acid sequence based on the hydrophilicity index or hydropathic index of the amino acids.

A variant polypeptide of the present invention has less than 100%, but at least about 50%, and more preferably at least about 80% to about 90% amino acid sequence homology or identity to the amino acid sequence of a corresponding native nucleic acid molecule or polypeptide comprising SEQ ID NO 1, SEQ ID NO 2, SEQ ID NO 3, or SEQ ID NO 4. The amino acid sequence of a variant MCPIP polypeptide therefore corresponds essentially to the disclosed amino acid sequences. As used herein, “corresponds essentially to” refers to a polypeptide sequence that will elicit a similar biological activity as that generated by a disclosed MCPIP, such activity being from at least about 70 percent of that of disclosed MCPIP polypeptide, to greater than 100 percent of the activity of a disclosed MCPIP peptide.

A variant of a disclosed MCPIP may include amino acid residues not present in the corresponding MCPIP, or may include deletions relative to the corresponding MCPIP. A variant may also be a truncated “fragment” as compared to the corresponding MCPIP, i.e., only a portion of the amino acid sequence of certain disclosed MCPIPs.

MCPIP is expressed in monocytes, vascular endothelial cells and cardiac myocytes and upregulates members of the apoptotic gene family involved in the induction of cell death. Chromatin immunoprecipitation revealed that MCPIP interacted with the N-cadherin 12/19 promoter, which has been shown to be associated with vascular stabilization by interacting with periendothelial cells during vessel formation Gerhardt, H. et al. Dev. Dyn. (2000) 218: 472-479; Luo, Y., et al. J. Cell Biol. (2005) 169: 29-34). These observations by the inventors led them to further investigate the potential involvement of MCPIP in angiogenesis. They modulated the expression of hMCPIP in human umbilical vein endothelial cells (HUVECs) and analyzed the angiogenic activity of hMCPIP, as indicated by its activation of expression of angiogenesis-related genes. In those studies, upregulation of MCPIP enhanced endothelial cell migration and capillary-like tube network formation, and increased the expression of angiogenesis-related genes. These effects were appropriately inhibited by hMCPIP-specific small interfering RNA (siRNA).

The inventors identified novel downstream targets of hMCPIP, cadherin-12 and cadherin-19. Knockdown of hMCPIP expression significantly reduced the rnRNA transcripts of cadherin-12 and cadherin-19. Furthermore, the angiogenic activity of MCP-1 was clearly attenuated by hMCPIP-specific siRNA, indicating that MCP1P is a novel angiogenic factor that may exert its function by regulating the expression of cadherin-12 and cadherin-19.

MCP-1 and its inducible protein MCPIP play a direct role in angiogenesis and neovascularization and therefore represent useful targets for promoting blood flow to ischemic cardiac and other tissues to treat cardiovascular disease and for inhibiting angiogenesis and tumor progression.

Consistent with its role in endothelial sprouting and tube formation, many of the genes identified to be up-regulated by MCPIP include molecules associated with cell communication and morphogenesis. These genes included the growth factors and receptors (PDGF-a, EGF, HIF 1-a, EphA1, EphA3, EphBZ), cytokines and chemokines (IL-I P, CSF-3, CXCL-2, CXCL-3, CXCL-9), adhesion molecules and matrix proteins (VE-cadherin, Thrombospondin-I, IL-8) as well as proteases and their inhibitors (MME′-9, TIMP-2, Plasmogen activator). Such genes are now recognized to modulate the biological processes of angiogenesis. For example, EphBZ is reported to be important in directed cell migration and branching development (Cheng, N., et al. Cytokine Growth Factor Rev. (2002) 13: 75-85) and EphA3 is important for adult neovascularization. Similarly, inflammatory cytokine IL-10 has been shown to be necessary for tumor angiogenesis. In particular, the notch homolog 4 is implicated in multiple aspects of vascular development (Iso, T. et al. Arterioscler. Thromb. Vasc. Biol. (2003) 23: 380-387). The inventors' Oligo GEArray (SuperArray Bioscience Corporation, Frederick, Md.) data demonstrate up-regulation of EphA3 (5.8-fold), EphB2 (8.6-fold), IL-0 (1 1.7-fold), and notch homolog 4 (11-fold) by MCPIP.

Cadherins are commonly activated by vascular remodeling-related molecules and play a central role in the initiation of cellular response and the assembly of the vascular network (Wheelock, M. J. and K. R. Johnson (2003) Annu. Rev. Cell Dev. Biol. 19: 207-235; Jain, R. K. (2003) Nat. Med. 9: 685-693). ECs express two major cadherins, VE- and N-cadherins. The importance of VE-cadherin in vascular development has been well established, whereas N-cadherin is thought to function in adherence junctions between endothelial cells and mural cells (pericytes and vascular smooth muscle cells) (Navarro, P. et al. (1998) J. Cell Biol. 140: 1475-1484). Although N-cadherin has been known to be abundantly expressed in endothelial cells (Salomon, D. et al. (1992) J. Cell Sci. 102: 7-17), its role in endothelial cell function, including angiogenesis, has remained elusive. The endothelial-specific knockout of N-cadherin in mice led to an aberrant vasculature both in the embryo and in the yolk sac, resulting in embryonic lethality at mid-gestation (Luo, Y. and G. L. Radice (2005) J. Cell Biol. 169: 29-34). Recently, N-cadherin has been found to play a fundamental role in angiogenesis by modulating adherence junction components and EC behavior (Luo, Y. and G. L. Radice (2005) J. Cell Biol. 169: 29-34). The inventors have obtained direct evidence for the effects of hMCPIP on cadherin-12 and cadherin-19 transcription, provided by chromatin immunoprecipitation assay, which demonstrated that hMCPIP interacted with the cadherin-12 and the cadherin-19 promoter. Interaction was dependent on the DNA-binding domain of hMCPIP, which was also confirmed by electrophoretic mobility shift assay. Moreover, hMCPLP gene transfer induced cadherin-12 and cadherin-19 promoter gene expression in HUVECs that was accompanied by HUVEC capillary-like tube formation, and this effect was significantly suppressed by hMCPIP-specific siRNA. Interestingly, cadherin-12-, and cadherin-19-specific siRNA also significantly attenuated HUVEC capillary-like tube formation induced by over-expression of hMCPIP.

The inventors have also determined that MCPIP is a cell death inducer that is involved in the development of ischemic heart disease in both an animal model and human cardiac tissue. MCPIP expression increases in parallel with progressive cardiac dysfunction. In situ hybridization showed MCPIP transcripts in cardiomyocytes and immunohistochemistry demonstrated that MCPIP was associated with cardiomyocyte nuclei. Realtime PCR analysis showed MCPIP transcript levels to be much higher in ischemic failing myocardium in humans than that of nonischemic myocardium, indicating that MCPIP is a useful target for preventive and therapeutic compounds and methods for ischemic heart disease.

Because MCPIP induces expression of a variety of genes in response to MCP-1, MCPIP activation, over-expression, gene transfer, protein delivery, inhibition of activation, gene-knockout, inhibition by siRNA, and inhibition of nuclear localization, for example, represent therapeutic opportunities for the treatment of a variety of diseases associated with MCP-1 and MCPIP. For example, where it is desirable to stimulate angiogenesis, as in wound repair, delivery of MCPIP protein via means such as, for example, delivery of one or more fusion proteins comprising MCPIP protein or a functional subunit or variant thereof and at least one cell-permeable peptide effective to promote transfer of MCPIP into a cell when delivered extracellularly, may provide an effective therapy.

In certain disease states, such as ischemic cardiovascular disease, peripheral artery disease, atherosclerosis and restenosis after balloon injury or stent implantation, for example, an effective therapy may comprise treatment of a subject by administering a therapeutically effective amount of a cell-permeable peptide additionally comprising a peptide having an amino acid sequence that will compete for binding with the nuclear localization sequence of MCPIP, thereby blocking nuclear localization of MCPIP. A similar type of peptide has been demonstrated to be effective in blocking nuclear localization of NF-kB. For example, a cyclic form of a peptide formed by the fusion of a cell-permeable sequence and the nuclear localization sequence of NF-kB has demonstrated in vivo efficacy in preventing LPS-induced liver apoptosis and death in mice (Liu, D. et al. J. Biol. Chem. (2004) 279(46): 48434-48442). Alternatively, a cell-permeable peptide may be operably linked to an amino acid sequence or a peptide-nucleic acid that will compete for and block binding to DNA in the nucleus by MCPIP.

Similarly, some authors have proposed that inhibition of MCP-1 signaling could be a new acute treatment approach to limit infarct size after stroke (J. Cereb. Blood Flow Metab. (2002) 22(3): 308-17). The inventors' discovery that MCPIP induces expression of a variety of genes that are involved in the inflammatory response and cell-death response associated with stroke offers an attractive method for treating stroke. For example, for an individual suspected of suffering a stroke a therapeutically effective amount of a composition comprising a cell-permeable peptide sequence functionally attached to a molecule that competitively blocks nuclear localization of MCPIP or binding of MCPIP to nuclear DNA could be administered via oral, intravenous, intraperitoneal, subcutaneous, or other means known to those of skill in the art, to limit the localized inflammation and increased infarct size associated with stroke.

As used herein, “inhibitors of MCPIP” or “MCPIP inhibitors” generally refer to compositions that produce inhibition of MCPIP induction, activation, nuclear localization, or induction of MCPIP-induced genes. These types of molecules may provide effective therapies for treating ischemic cardiovascular disease, cancer, tuberculosis, sarcoidosis, and a variety of other diseases for which there is a significant inflammatory, particularly chronic inflammatory, component. Where MCPIP is associated with the promotion of the disease state, inhibition of MCPIP activity, DNA-binding, nuclear localization, etc., may be a therapeutic option for preventing or treating the disease. The inventors have demonstrated that siRNA can be used to inhibit the effects of MCPIP and achieve the desired outcome in the cell.

One or more MCPIP inhibitors may be used in conjunction with balloon angioplasty to decrease restenosis and atherosclerosis following the procedure. Such MCPIP inhibitor compositions may be administered locally by means such as, for example, via a pharmaceutical pump to provide one or more MCPIP inhibitor compositions to the immediate area or may be administered systemically for a period of time following the procedure to decrease restenosis and atherosclerosis. MCPIP inhibitors may also be administered in conjunction with implantation of a cardiovascular stent. Administration may be provided by coating the stent with one or more MCPIP inhibitors, by implanting a pharmaceutical depot within the tissues adjacent to the stent for release of the MCPIP inhibitor(s) into the stent area, or by providing to a patient an oral, intravenous, intraperitoneal, or other dose of MCPIP inhibitor(s) for more systemic administration.

MCPIP inhibitors, such as, for example, a cell-permeable peptide comprising the nuclear localization sequence of MCPIP, may be utilized to decrease angiogenesis for the purpose of inhibiting tumor progression. Similarly, one or more MCPIP inhibitors may be used to treat certain tumors such as hemangioendotheliomas, blood vessel tumors that cause facial deformities in infants and young children. Depending upon the site of the tumor, an MCPIP inhibitor may be provided locally or systemically.

Ischemic retinopathy is a major cause of blindness worldwide, has also been associated with elevated levels of MCP-1. For the treatment of retinopathies, one example of in MCPIP inhibitor composition for therapeutic use is a cell-permeable protein or peptide comprising an inhibitor of nuclear localization of MCPIP. Such a composition may be provided as eye drops for administration to an individual who is either at risk for the development of ischemic retinopathy, such as an individual with diabetes, or an individual who has been diagnosed with ischemic retinopathy. Modified release compositions comprising MCPIP inhibitor compositions may also be used. Such compositions may comprise, for example, polymer-coated spheres, nanoparticles, or other delivery vehicles known to those of skill in the art of pharmaceutical formulation.

Increased MCP-1 expression in adipocytes has been associated with both Type 1 and Type 2 diabetes, white adipose tissue is a major source of MCP-1, and MCP-1 has been shown to be an insulin-responsive gene (Sartipy & Loskutoff (2003) Proc. Natl. Acad. Sci. USA 10.1073/pnas.1133870100; Diabetologia (2001) 44(3): 325-332; Mol. Cell. Biochem. (2005) 276(1-2): 105-111). MCP-1 impairs insulin signaling in skeletal muscle cells at doses similar to its physiological plasma concentrations (Sell, H. et al. (2006) Endocrinology 147(5): 2458-2467). Inhibiting the action of MCP-1 produced by adipose tissue cells as fat is accumulated in the body has therefore been proposed to be one therapeutic approach to preventing or treating diabetes. The inventors have demonstrated that MCPIP levels are elevated in mice as they gain weight, increase white adipose tissue, and increase fasting glucose levels as the result of a high-fat diet. Targeting MCPIP is therefore an attractive target for diabetes treatment and prevention. Weisberg, et al. (Weisbert, S. P., et al. J. Clin. Invest. (2006) 116(1): 115-124) demonstrated that CCR-2 dependent pathways could be affected sufficiently by a CCR2 antagonist to permit detectable differences in insulin sensitivity in obese mice after 2-3 weeks of treatment. As has been demonstrated in vivo with cell-permeable peptides that block nuclear localization of NF-kB and therefore block its effects, a cell-permeable peptide may be used to block nuclear localization or DNA binding of MCPIP and therefore block its effects. siRNA may also be used to inhibit the effects of MCPIP.

MCPIP, as an MCP-1-inducible protein, may also have application as an accelerator of wound healing, as MCP-1 has been shown to accelerate wound healing (Low, Q. E., et al. Am. J. Pathol. (2001) 159: 457-463). Similarly, MCP-1 has been demonstrated to be a major factor in the development of diseases such as scleroderma rheumatoid arthritis, multiple sclerosis, asthma, inflammatory bowel disease, and systemic lupus erythematosis (SLE). MCPIP inhibitors, competitors, and antagonists provide a therapeutic option for the treatment of these diseases.

MCP-1 and CCR2 have been associated with organ and tissue fibrosis, such as lung fibrosis and kidney fibrosis (Kitagawa, K. et al., Am. J. Pathol. (2004) 165(1): 237-246). A decrease in MCP-1 has been associated with improved kidney morphology and function in patients with kidney failure (Nephrol. Dialysis Transplant. (1997) 12(3): 430-437). One or more MPCIP inhibitors may therefore be of value in the treatment of kidney disease. CCR2 regulates recruitment and activation of lung fibrocytes after respiratory injury (Moore, B. B. et al., Am. J. Pathol. (2005) 166(3): 675-684). Modulating the level of recruitment and activation of lung fibrocytes by inhibition of MCPIP provides an option for preventing lung fibrosis after respiratory injury.

MCP-1 is associated with neurological damage induced by viral infection. MCPIP inhibitors provide an option for treating virally-induced neurological damage, especially the neurological damage associated with Human Immunodeficiency Virus (HIV). MCP-1 and CCR2 have been associated with inflammatory pain and chronic pain, blockade of the CCR2 receptor having been suggested as a therapy for treatment of chronic pain (Abbadie, C., et al. Proc. Natl. Acad. Sci. USA (2003) 100(13): 7947-52). Inhibiting activation, nuclear transport, or DNA binding of MCPIP, for example, may provide an even more effective option for treatment of inflammatory and/or chronic pain.

The invention also provides methods for identifying pharmaceutical compositions that enhance or inhibit the effects of MCP-1 or MCPIP. In one embodiment, a method for identifying a pharmaceutical composition that enhances the effect of MCP-1 comprises applying the pharmaceutical composition to cultured cells and comparing the level of cellular MCPIP as compared to a control to which no pharmaceutical composition is applied, enhancers of MCP-1 being identified as those compositions that increase cellular levels of MCPIP. In another embodiment, a method for identifying an inhibitor of MCPIP comprises administering to a cell a pharmaceutical composition and determining the level of MCPIP translocation to the nucleus as compared to that of control cells that are untreated with the composition. In another embodiment a method may comprise administering a pharmaceutical composition to a cell and determining the presence, absence, or relative level of DNA binding of MCPIP as compared to that of an untreated control.

The invention may be further described by means of the following non-limiting examples.

### Examples

### Cell Culture Conditions

Human umbilical venous endothelial cells (HUVECs) were obtained from Clonetics (Cambrex Bio Science, Walkersville, Inc, MD). HUVECs were grown in endothelial cell basal medium supplemented with hydrocortisone (1 μg/ml), bovine brain extract (12 μg/ml), gentamicin (50 μg/ml), amphotericin B (50 ng/ml), epidermal growth factor (10 ng/ml), and 2% fetal bovine serum (EGM SingleQuots®, Clonetics/Cambrex) as recommended by the manufacture. HUVECs were used between passages 4 and 8. All cells were maintained at 37° C. in 5% CO₂.

### Plasmid Construction and Transfection

The human MCPIP (hMCPIP) cDNA encoding the full-length human MCPIP (GenBank accession number AY920403) was cloned into BamH1 and EcoR1 sites of a pEGFP/N1 vector to generate Green Fluorescent Protein (GFP)-tagged hMCPIP. Transient transfection of hMCPIP plasmid in HUVECs was performed using LipofectAMINE PLUS Reagent (Life Technologies, Inc) with a transfection efficiency of about 60-70%, as determined by over-expressing GFP and counting the percentage of cells showing green fluorescence compared to the total cell number.

### RNA Interference Experiments

HUVECs, 4th generation, were cultured in EGM BulletKit® medium (CC-3124, Cambrex) according to the manufacturer's recommendations. For RNA interference analysis, human MCPIP siRNAs targeting the sense sequence 5′-3′ and the anti-sense sequence 5′-3′ (each -pmol) [human MCPIP SMARTpools (Dharmacon) were delivered into 70% confluent cells with transfection at the final concentration of 50 nM according to the manufacturer's protocol] selected and incubated in 200 μl OPI medium in the presence of 6 μl Lipofectamine (Invitrogen) for 30 minutes at room temperature. HUVECs (5×10⁴ cells/per well) were washed with OPI medium and incubated with OPI medium containing lipofectamine/siRNA mixture (final concentration 50 nm of siRNA) for 6 hours. 2 ml of fresh EBM complete medium were added and the cells were incubated for 24 hours.

### Cell Migration Assays

5×10⁴ HUVECs per well were seeded into 6-well plates and grown to confluence. Cells were transfected with pEGFP/hMCPIP, pEGFP/hMCPIP plus siRNA, and the empty vector pEGFP/N1 as described above. Six hours later after transfection, the cell monolayer was wounded with a plastic pipette tip to generate a wound with a width of approximately 1 mm. The remaining cells were washed twice with culture medium to remove cell debris and incubated at 37° C., 5% CO₂ for 24 hours. The number of cells that had migrated across the edge of the wound and into the denuded area (as indicated with black box outline) was photographed and counted as migrating cells using the Metamorph Series 6.2 image program (Universal Imaging, West Chester, Pa.). Results were expressed as the average number of cells per field of view. The experiment was repeated three times.

### BrdU Incorporation Assay

To determine the effect of MCPW on cell proliferation, the rate of DNA synthesis was established by measuring BrdU incorporation in control and HUVECs transfected with MCPIP or plus siRNA in 8-well chamber glass slides. After incubation for 6 hours with 10 μM BrdU, cells were fixed for 10 min with 3.7% paraformaldehyde and stained with an anti-BrdU antibody (Novus) for 60 min at room temperature. The antibody was washed off with DPBS and the secondary anti-rat IgG Cy₂ (fluorochrome) antibody 0 was added at a dilution 1:500 for 30 min at 37° C., and then washed with DPBS. Slides were mounted for viewing under the fluorescence microscopy and the percentage of BrdU-positive nuclei (red) was measured by counting five randomly selected fields under 20* magnification using the Metamorph Series 6.2 image program (as above). The experiment was repeated three times.

### In Vitro Capillary-Like Tube Formation Assays

Nine volumes of ECMatrix™ gel solution and one volume of ECMatrix™ diluents buffer (Chemicon International, Inc., USA) were mixed on ice. A volume of 50 μl of the ECMatrix™ mixture was dispensed into a well of a pre-cooled 96-well tissue culture plate and the matrix solution allowed to solidify for 1 h at 37° C. before cell seeding. Transfected HUVECs (1×10⁴ cells/per well) were added onto the surface of the polymerized ECMatrix™ and incubated in EBM complete medium for 24 h. Tube formation was observed under phase-contrast microscopy and photographed. Tube formation ability was quantified by counting the total number of cell clusters and branching in five randomly chosen microscopic fields per well under 100 * magnification. Results were expressed as the mean percentage of branching over total cell clusters, and expressed as a ratio to the control. The experiment was repeated three times.

To examine the contribution of MCPIP to MCP-1-induced angiogenic activity, cells were incubated in EBM medium with the presence or absence of siRNA and the recombinant mouse MCP-1 (100 ng/ml) was added to the medium for 24 hours. Tube formation was observed under phase-contrast microscopy and photographed and measured as described above.

Detection of Apoptotic Cells by 4,6-diamidino-2-phenylindole (DAPI) Staining

Apoptotic cells were detected by DAPI nuclear staining according to the procedure described previously King M T, et al. (Oncogene (2003) 22:4498-4508). Briefly, 5×10⁴ HUVECs per well were seeded onto 4-well chamber glass slides and were grown to confluence. Cells were transfected with pEGFP/hMCPIP or the empty vector pEGFP/N 1 as described above for 24 hours. The medium was removed and DAPI was added at a 2 μg/ml dilution and allowed to incubate with the cells for 10 min. The DAPI (4,6-diamidino-2-phenylindole, Sigma Chemical Co.) was removed and the cells were fixed with 3.7% paraformaldehyde and washed 3 times with DPBS at room temperature. Slides were mounted for viewing under fluorescence microscopy. Cells undergoing apoptosis stained strongly for DAPI as compared to attached living cells. The number of cells positively stained were counted and divided by the total number of cells in ten randomly selected fields of view. The experiment was repeated three times.

### Gene Expression Profiling by Oligo GEArray Microarray

Angiogenesis-related gene expression profiling was performed using OligoGEArray human angiogenesis microarray which contained a total of 117 different genes, including the growth factors and receptors, cytokines and chemokines, adhesion molecules and matrix proteins, as well as proteases and their inhibitors involved in modulating the biological processes of angiogenesis. HUVECs were seeded in 25 cm² flasks at a density of 2.0×10⁵ cells/flask and transfected with pEGFP/hMCPIP, pEGFP/mMCPIP plus siRNA or the empty vector pEGFP/N, then incubated for 24 hours as described above. Total RNA was prepared from HUVECs transfected with pEGFP-N1 or pEGFP-hMCPIP and poly (A)+RNA was purified from the total RNA by using an Oligotex-dT30 mRNA purification kit.

### Chromatin Immunoprecipitation

HEK 293 cells (3×10⁷), transfected with pEGFP/N1 or pEGFP/hMCPFP vector, were cross-linked for 10 minutes by adding 1% formaldehyde to the DMEM medium. The fixed cells were washed with PBS and then were lysed with lysis buffer (10 mmol/L EDTA, 1% SDS, 1 mmol/L PMSF, 1 μg/ml pepstatin, 1 μg/ml leupeptin, 1 μg/ml aprotinin, 50 mM Tris/HCL, pH 8.1) and sonified 4 times for 15 seconds with output 3 (Branson Sonifire 450, Branson). For chromatin immunoprecipitation, cell lysates were incubated with a rabbit polyclonal antibody against hMCPIP (Zhou et al). The isolated precipitated DNA was introduced in TOPO plasmid vector using Zero Blunt TOPO PCR cloning kit according to the manufacturer's instructions (Invitrogen), and was amplified by PCR with primers corresponding to a 529 bp fragment of the human cadherin-12 promoter (forward), and a 1808 bp fragment of the human cadherin-19 promoter (forward).

### Electrophoretic Mobility Shift Assays

Total cell extracts were prepared from HEK293 cells transfected with pEGFPM1 or pEGP/hMCPIP vector as described above. Binding reactions were performed in a 25 ul volume containing purified hMCPIP protein (0.4 μg) and ³²P-labeled probe. Non-denaturing polyacrylamide gels (4%) were electrophoresed at 40° C. for 4 hours and were exposed to X-ray film for 24 hours.

### Reverse Transcription-Polymerase Chain Reaction (RT-PCR) Analysis

Total RNAs from HUVECs transfected with pEGFP-NI, pEGFP-MCPIP or siRNA were isolated. The extracted RNA was converted to single-strand cDNA using an RT-PCR system followed by PCR amplification. Each RT-PCR reaction consisted of 25-30 cycles at 94° C. for 30 s, 60° C. for 30 s, 72° C. for 30 s and final extension at 72° C. for 10 min. Human cadherin-12 primers were used for amplification. P-actin was used as an internal control. PCR products were electrophoresed on 1.5% agarose gel stained with ethidium bromide.

To examine whether MCP-1-induced angiogenic activity up-regulates cadherin expression via MCPIP, the cells were incubated in EBM medium with the presence or absence of siRNA and the recombinant mouse MCP-1 (100 ng/ml) was added to the medium for 24 hours. The expression of cadherins was detected by RT-PCR as described above.

Overexpression of hMCPIP in ECs Induces Capillary-Like Tube Formation

To evaluate the potential role of MCPIP in angiogenesis, the inventors examined the effects of MCPIF on angiogenesis by testing capillary-like tube formation in HUVECs. After transient transfection with hMCPIP in HUVECs for 24 hours, the increased expression of hMCPIP mRNA was confirmed by real-time PCR analysis. To determine whether the elevated expression of hMCPIP increases capillary-like tube formation of vascular endothelial cells in vitro, the inventors planted transfected HUVECs (1×10⁴ cells 1 per well) onto the surface of the polymerized ECMatrix™ in a 96-well plate. After 24 hours of incubation on ECMatrix™ the inventors observed an increase in the number of network structures of capillary when compared with control plasmid transfected HUVECs.

To determine whether upregulation of hMCPIP transcript is actually cortical for capillary-like tube formation, the inventors developed a siRNA method to specifically suppress hMCPIP expression in HUVECs. Knockdown of hMCPIP transcripts with hMCPIP-specific siRNA markedly inhibited HUVEC capillary-like tube formation.

Effects of hMCPIP on Angiogenesis-Related Properties of HUVECs

Capillary-like tube formation in three-dimensional fibrin gels depends on vascular permeability as well as on the invasive, migratory, and proliferative potential of endothelial cells. This process begins with the formation of endothelial cell sprouts initiated by apoptosis followed by the proliferation and migration of neighboring endothelial cells along preformed extensions [20]. The inventors infected HUVECs with hMCPIP or the control vectors and examined their effects on angiogenesis-related properties of HUVECs. After 24 incubation, as assessed by wound assays, HUVECs transfected with hMCP1P displayed significantly increased cell migration as compared to cells transfected with control vectors. The inventors compared DNA synthesis, as determined by BrdU incorporation, in control HUVECs and HUVECs transfected with hMCPIP. No differences in DNA synthesis were observed between control, GFP expressing, and hMCPIP-GFP expressing HUVECs. DAPI staining was performed in cultured HUVECs to detect cell apoptosis after transfection with hMCPIP or control vector. After 24 hours, HUVECs transfected with hMCPIP showed a high number of DAPI-positive cells as compared to cells transfected with control vectors. Knockdown of hMCPIP transcripts with MCPIP-specific siRNA significantly inhibited hMCPIP transfection-induced cell death and migration of HUVECs, respectively. These findings indicate that hMCPIP is responsible for the induction of angiogenesis-related properties of HUVECs.

Profiling of Gene Expression in HUVECs Transfected with MCPIP

hMCPIP has transcription factor characteristics, so the inventors performed microarray analysis using Oligo GEArray human angiogenesis microarrays to detect changes in the expression of 113 human angiogenesis-related genes which include the growth factors and receptors, cytokines and chemokines, adhesion molecules and matrix proteins, as well as proteases and their inhibitors that are involved in modulating the biological processes of angiogenesis. Genes were considered up- or down-regulated when the averaged expression level (hMCPIP/GFP control) was 2.0-fold above or 0.5-fold below, respectively. When RNA harvested from control vectors- and hMCPIP-expressing HUVECs was hybridized, the inventors observed upregulation of 29 of 113 angiogenesis-related genes in HUVECs transfected with hMCPIP as compared to cells transfected with control vectors (Table 1). These up-regulated genes included ephrin A 1, ephrin B2, ephrin A3, IL-1β, notch homolog 4, angioprotein-2, neuropilin-1, plasminogen activator, PDGF-A, TIMP-2, MMP-9, and chemokine ligands. To test if these genes represented specific targets of MCPIP, the inventors performed RNA interference experiments following transfection with MCPIP-specific siRNA. Microarray analysis revealed that expression of most of these upregulated genes, such as ephrin A 1, ephrin B2, ephrin A3, IL-1β, neuropilin-1, notch homolog 4, angioprotein-2, TIMP-2, MMP-9, and chemokine ligands, were inhibited (<0.5-fold) or significantly abrogated by hMCPIP-specific siRNA transfectants.

TABLE 1
Expression Profile of Angiogenesis-related Genes
in GFP/MCPIP - Over GFP-Infected HUVECs

| Gene Name | Fold Induction |
|---|---|
| Ephrin A1 | 12.0 |
| Interleukin-1β | 11.7 |
| Notch Homolog 4 | 11.0 |
| Ephrin B2 | 8.6 |
| PDGF-A | 7.6 |
| TIMP-2 | 6.8 |
| Ephrin A3 | 5.8 |
| MDK | 5.1 |
| Thrombospondin-1 | 5.0 |
| Colony Stimulating Factor-3 | 5.0 |
| Angioprotein-2 | 4.4 |
| Chemokine (CXC motifs) Ligand-9 | 4.3 |
| Angiogenic factor with FHA Domains | 4.0 |
| MMP-9 | 3.8 |
| Hypoxia Inducible Factor-1 | 3.6 |
| Chemokine (CXC motifs) Ligand-2 | 3.5 |
| Chemokine (CXC motifs) Ligand-3 | 3.4 |
| Chemokine (C-C motif) Ligand-11 | 3.2 |
| Epidermal Growth Factor | 3.2 |
| Neuropilin-1 | 3.1 |
| Collagen type IV-α3 | 2.6 |
| Angioprotein-1 | 2.5 |
| Chemokine (C-C motif) Ligand-2 | 2.5 |
| TNF Superfamily 12A | 2.5 |
| Angioprotein like-4 | 2.4 |
| Chemokine (CXC motifs) Ligand-1 | 2.4 |
| uPA | 2.2 |
| Interleukin-8 | 2.0 |
| Jag1 | 2.0 |

hMCPIP Transcriptionally Activates the Cadherin-12 and Cadherin-19 Promoters.

Traditionally, transcription factors are localized in the nucleus where they bind, either directly or indirectly to the DNA and take part in the induction or inhibition of gene transcription. To clarify the potential targets for hMCPIP, chromatin immunoprecipitation assays was performed in HEK 293 cells transfected with hMCPIP or control vectors for 12 hr. After cross-linking, the DNA recovered from immunoprecipitates of hMCPIP was sequenced. The DNA sequences that bound MCPIP in vivo were found to be located in the genes that encode cadherin-12 and cadherin-19 showing that these genes are the direct targets of MCPIP. This finding was confirmed by the finding that the expression of cadherin-12 and cadherin-19 was increased in HEK293 cells transfected with hMCPIP while expression of J3-actin was at the same levels in cells transfected with hMCPIP or control vectors. The identification of the amplified immunoprecipitated DNA fragments was confirmed by electrophoretic mobility shift assay showing specific binding of MCPIP. These results suggest that hMCP1P interacts with the cadherin-12 and cadherin-19 promoters. Cadherins have been shown to play a central role in the initiation of the cellular response and the assembly of the vascular network. Because cadherin-12 and cadherin-19 are potential targets of hMCPIP, the inventors analyzed the regulation of cadherin-12 and cadherin-19 by hMCPIP in HUVECs during angiogenesis. Transfection with HMCPIP in HUVECs significantly increased the cadherin-12 and cadherin-19 transcript sas compared to cells transfected with control vectors. In contrast, knockdown of hMCPI expression by MCPIP-specific siRNA significantly reduced the expression of cadherin-12 and cadherin-19, suggesting that hMCPIP indeed upregulates expression of cadherin-12 and cadherin-19 in HUVECs. siRNA-mediated knockdown of hMCPIP attenuates MCP-1-induced angiogenic activity.

MCP-1 has been recognized as an angiogenic chemokine. To test whether MCPP mediates MCPI-1 induced angiogenesis, the inventors developed a siRNA method to specifically suppress MCPLP expression in HUVECs. RT-PCR analysis revealed the increased levels of MCPIP mRNA transcripts in HUVECs after treatment with MCP-1. Transfection with a MCPIP-specific siRNA in MCP-1-treated HUVECs resulted a marked down-regulation of MCPIP mRNA transcripts. HUVECs treated with MCP-1 showed significantly increased capillary-like tube formation by 59% over control (P<0.001), and this increase was significantly blunted nearly to basal levels by transfection with MCPIP-specific siRNA (P<0.001).

The inventors also examined the effects of downregulation of MCPIP on the expression profile of MCP-1-induced angiogenesis-related genes using Oligo GEArray human angiogenesis microarray. Genes were considered up- or down-regulated when the averaged expression level (MCPIP control) was 2.0-fold above or 0.5-fold below, respectively. As summarized in Table 2, 32 of 113 angiogenesis-related genes were upregulated in MCP-1-treated HUVECs as compared to untreated-HUVECs. These up-regulated genes include growth factors and receptors (PDGF-B, angioprotein-like 3, VEGF family, endothelial cell growth factor-1), adhesion molecules (endoglin, VE-cadherin, endostatin, shingolipid G-protein coupled receptor-1, IL-8), proteases and their inhibitors (angioprotein-like 4, PECAM-1, MMP-2, TIMP-1) and others (ephrin A2, TEK, plasmogen activator, chemokine ligands) that involved in modulating the biological processes of angiogenesis. When cells were treated with hMCPIP-specific siRNA, most of these up-regulated angiogenesis-related genes were knocked down or markedly suppressed by hMCPIP-specific siRNA (Table 2).

The inventors next examined whether MCP-1 promotes increased expression of cadherin-12, and cadherin-19 in HUVECs during the development of tube formation. RT-PCR analysis revealed a significant increase in cadherin-12 and cadherin-19 mRNA transcripts in HUVECs after treatment with MCP-1 for 24 hours, and this increase was markedly suppressed by infection with hMCPIP-specific siRNA.

TABLE 2
Expression Profile of Angiogenesis-related Genes in MCP-1 − and
MP-1 + MCPIP-specific siRNA-treated HUVECs

| | MCP-1 | siRNA + MCP-1 |
|---|---|---|
| Gene Name | Fold Induction | |
| Angioprotein-like 3 | 10.0 | |
| Angioprotein-like 4 | 5.0 | |
| Cadherin 5 | 5.0 | |
| CD13/GP156 | 5.0 | |
| Endoglin | 5.0 | |
| Chemokine (CXC motifs) Ligand-11 | 5.0 | 2.4 |
| Sphingolipid G-protein coupled receptor-1 | 5.0 | 2.4 |
| Laminin-α5 | 4.7 | 2.1 |
| TIMP-1 | 4.7 | |
| Prostaglandin endoperoxide synthase | 4.6 | 5.0 |
| Endostatin | 4.6 | 2.1 |
| AKT-1 | 4.2 | 2.4 |
| PECAM-1 | 4.2 | |
| Tie-1 | 4.2 | |
| VEGF-C | 4.1 | |
| Thrombospondin-1 | 4.1 | 2.5 |
| MMP-2 | 3.9 | 2.0 |
| Endothelial cell growth factor-11 | 3.5 | 3.6 |
| VEGF-B | 3.5 | |
| Chemokine (CXC motifs) ligand-10 | 3.4 | 4.7 |
| Angioprotein-1 | 3.2 | |
| Tie-2 | 3.2 | |
| PDGF-B | 3.1 | 2.0 |
| IL-8 | 2.7 | |
| Ephrin A2 | 2.5 | |
| Jag-1 | 2.3 | 2.2 |
| Chemokine (CXC motifs) ligand-2 | 2.1 | 2.4 |
| Epidermal Growth Factor | 2.0 | |
| Chemokine (CXC motifs) Ligand-1 | 2.0 | |

### Induction of Apoptotic Genes by MCPIP Expression in HEK293 Cells

To determine whether expression of MCPIP causes detectable upregulation of genes known to be involved in cell death, microarray analysis was done with RNA isolated soon after MCPIP expression was clearly indicated by the fluorescence of the fused GFP (16 hours) but before cell death was detectable. This gene expression profile showed that MCPIP caused induction of several genes the products of which are known to be involved in cell death (Table 3).

TABLE 3
Upregulated Genes After MCPIP Overexpression in HEK293 Cells

| | | Fold | |
|---|---|---|---|
| Gene Family | Genes | Expression | Description |
| Apoptotic | Bar | 17.3 ± 1.53 | Bifunctional apoptosis |
| regulator | | | regulator |
| | TNFR2 | 37.9 ± 5.6 | Tumor necrosis factor |
| | | | receptor Superfamily1B |
| BCL2 | BAX | 2.0 ± 0.03 | BCL2-associated X |
| | | | proteins |
| | BCL2L1 | 6.2 ± 0.99 | BCL2-like 1 |
| | BNIP3L | 2.0 ± 0.07 | BCL2/adenovirus E1B |
| | BCL2L9BOK | 8.4 ± 0.53 | BCL2-related ovarian |
| | | | killers |
| CARD | Apaf1 | 2.0 ± 0.07 | Apoptotic protease |
| | | | activating factor |
| Caspase | Casp9 | 6.3 ± 0.43 | Caspase-9 |
| CIDE | DFF40 | 3.4 ± 0.71 | DNA fragmentation |
| | | | factor β |
| Death | CRADD | 2.2 ± 0.06 | CASP2 and RIPK1 domain |
| domain | | | Containing adaptor with |
| | | | death domain |
| | DR3 | 2.3 ± 0.28 | Tumor necrosis factor |
| | | | receptor Superfamily 25 |
| Tumor | LTBR | 13.9 ± 0.84 | Lymphotoxin β receptor |
| necrosis | | | (Tumor necrosis factor |
| factor | | | receptor super family 3) |
| receptor | TNFRS10A | 8.7 ± 0.56 | Tumor necrosis factor |
| | | | receptor superfamily 10A |
| | TNFRS10C | 19.5 ± 1.48 | Tumor necrosis factor |
| | | | receptor superfamily 10C |
| TRAF | TRAF3 | 7.9 ± 0.87 | Tumor necrosis factor |
| | | | receptor-associated |
| | | | factor 3 |
| | TRAF-5 | 2.1 ± 0.10 | Tumor necrosis factor |
| | | | receptor-associated |
| | | | factor 5 |

### Tissue Samples for HMCPIP Analysis in Ischemic Hearts

Human heart tissues were obtained from the explanted hearts of patients undergoing heart transplantation in The Ohio State University hospital. The patient data were kept confidential except for age and diagnosis as ischemic or nonischemic. All animal and human materials used were in accordance with the approval of Institutional Review Boards and Animal Use Committees.

### Purification of Human Monocytes.

Human monocytes were isolated from buffy coat preparations obtained from the American Red Cross. Using Ficoll-Plaque PLUS (Arnersham Pharmacia Biotech AB) and by further purification using an indirect magnetic labeling system and a monocyte isolation kit. Flow cytometry using double staining with antibodies CD14-FITC and CD45-PE showed >90% purity.

Cloning of Human MCPIP from Human Monocytes after Treatment with MCP-1.

Monocytes were treated with 7 nM MCP-1 and harvested at various intervals. Total RNA was isolated from the cells with Trizol reagent (Gibco, Grand Island, N.Y.) and cDNA was prepared using Moloney Murine Leukemia Virus Reverse Transcriptase (GIBCO, Grand Island, N.Y.). The hMCPIP cDNA was prepared by PCR using the total cDNA as template and the following primers: 5′-CGCATATGAGTGGCCCCTGTGGAG-3′ (sense) (SEQ ID NO: 5), and 5′-CGGGATCCTTACTCACTGGGGTGCTGG-3′ (antisense) (SEQ ID NO: 6). A PCR product of the expected size was recovered, ligated into the vector pCR2.1 (Invitrogen, Carlsbad, Calif.), and the ligation reaction mixture was used to transform to TOPO10 competent cells. Recombinant colonies were screened, and inserts were sequenced to confirm the absence of any mutations.

Expression of Human MCPIP in E. coli and Preparation of Polyclonal Antibody.

The ORF of hMCPIP excised from pCR2.1/hMCPP by digestion with BamHI and NdeI, was ligated to the C-terminal of His₁₀-Tag of the expression vector pET16b and expressed in E. coli BL21. Rabbit polycolonal antibody was prepared using SDS-PAGE gel segments containing the recombinant hMCPIP as previously described (Guo, et al. Arch. Biochem. Biophys. (1995) 323: 352-360).

### In Situ Hybridization.

A 406-bp cDNA fragment from mMCPIP ORF (from 403-809 bp) and 352 bp fragment from CCR2 ORF (from 722 to 1073 bp) were generated by PCR with specific primers, cloned into dual-promoter vector pCRII, and the ligation reaction mixture was used to transform competent cells of TOPO10. The recombinant plasmids were linearized with restriction enzyme Kpn I and used as template for in vitro transcription with RNA polymerase and digoxigenin (DIG)-labeled uridine-triphosphate using a dig RNA Labeling Kit (Roche, Indianapolis, Ind.). Frozen OCT compound-embedded sections were hybridized with DIG-labeled RNA probes (anti-sense or sense) and processed using standard procedures with anti-DIG antibodies conjugated alkaline phosphatase.

### Construction of Mutant MCPIP.

Standard PCR methods were used to generate the mutants. The nuclear localization signal (NLS) sequence RKKP was mutated to GGGP, the two conserved amino acids KC, within zinc finger motif, was changed to GG. PCR products were ligated to the N-terminus of EGFP within the vector pEGFP-N1 by using EcoR I and BamH I, and used to transform TOPO10 competent cells. Deletion of proline-rich regions were created using QuikChangeB Site-Directed Mutagenesis Kit from Stratagene. All substitution and deletion mutants were confirmed by sequencing.

### Cell Culture, Transfection, and Measurement of Cell Death and Viability.

HEK293 cells, grown in DMEM supplemented with 10% FBS, 1% Penicillin and Streptomycin, in a 5% CO₂-humidified atmosphere at 37° C., were transiently transfected with the plasmids pEGFP/hMCPIP or its mutants or control pEGFP-N1 using LipofectAMINE 2000 Transfection Reagent (Gibco, Grand Island, N.Y.). The day before transfection, HEK293 cells were plated in a 12-well plate at 4×10⁵ cells per well in 1 ml of DMEM supplemented with 10% FBS and 1% non-essential amino acids. For each well of cells, 1.6 μg plasmid DNA was combined with 4 μl of LipofectAMINE 2000 and incubated at room temperature for 20 min. DNA-LipofectAMNE 2000 reagent complex was added to each well. After 30 h cells were stained with propidium iodide and examined with a confocal microscope (MRC-600 Series Laser Scanning Confocal Imaging System, BioRad). Cell viability and death were measured by Trypan blue and TUNEL assays using standard procedures.

### In Vitro Assay for Transcription Factor Activity.

To elucidate the transcription factor activity of hMCPIP and its mutants, the inventors constructed a fusion protein of GAL4DNA binding with MCPIP and its mutants as test plasmid. The reporter plasmid had five GAL4 binding sites linked to firefly luciferase gene and in the positive control, activating transcription factor 4, was used to activate the luciferase gene. Experiments were repeated at least three times.

### Immunohistochemistry.

Immunohistochemistry was performed with paraffin-embedded sections. Antigens were retrieved using target retrieval solution (Pharmingen, San Diego, Calif.), sections were blocked in 3% hydrogen peroxide, incubated with polyclonal rabbit anti-human MCPIP antibody prepared as described above or isotype control overnight at 4° C., then incubated with horseradish peroxidase-conjugated goat anti-rabbit antibody (Santa Cruz Biotechnology, Santa Cruz, Calif.), visualized with diaminobenzidine and sections were counterstained with haematoxylin.

### Apoptosis Microarray Analysis.

Non-radioactive human apoptosis oligo microarrays (SuperArray Biosciences Corp. Frederick, Md.) were used. HEK293 cells were transfected (lipofectamine) with MCPIP-GFP or GFP alone as control for 6 hrs in serum free medium and then incubated in complete medium for 16 hrs before the isolation of RNA. Expression profiling was done according to the manufacturers instructions.

### Statistical Analysis.

Experimental data were analyzed using SPSS statistical software (SPSS Inc., Chicago, Ill., USA) in Windows XP. All values are presented as mean±SEM. Real-time PCR data were expressed as fold up-regulation compared with sex- and age-matched wild-type controls. Results were compared between groups by ANOVA analysis followed by t-tests. Differences were considered significant at P value of <0.05.

### Experimental Results

Treatment of human peripheral blood monocytes with MCP-1 resulted in transcriptional activation of a variety of genes including those which encode a variety of cytokines and chemokines, extracellular matrix degrading enzymes, cell adhesion proteins and a set of ESTs. The most highly induced EST, representing unidentified genes, was matched with a human cDNA clone with GeneBank accession number AW206332 which maps to a gene for a novel protein (FLJ23231) of unknown function on chromosome 1p^{33-35.3}. BLAST of the EST sequence against databases from NCBI and Celera showed homologous regions in the human genomic DNA. BCM Genefinder (http://.imgen.bcm.tmc.edu:9331/gene/gt.html) was used to predict the exons and the open reading frame (ORF). Databases from NCBI and Celera showed that the human MCPIP gene was 8.9 kb in length and contained 5 exons and 4 introns.

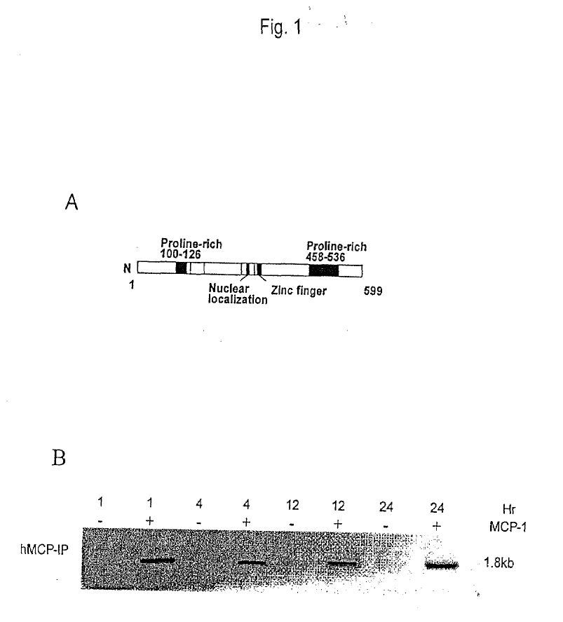

RNA from human peripheral blood monocytes treated with MCP-1 was used to perform RT-PCR to generate cDNA representing hMCPIP. The nucleotide sequence of the cloned cDNA showed an ORF that would encode a protein containing 599 amino acids with a calculated mass of 65.8 kDa (GenBank accession number AY920403). Protein motif analysis showed that MCPIP contains two proline-rich potential activation domains (FIG. 1A), one between residues 100 and 126 with 37% proline residues and the other at 458 to 536 with 28% proline residues. It also contains a monopartite nuclear localization signal sequence (RKKP) and a putative single zinc finger motif. Thus, MCPIP has features characteristic of a transcription factor.

Mouse genome data search revealed a gene highly homologous to the human mcpip gene. RT-PCR of mRNA isolated from a six-month-old MCP mouse heart gave the murine MCPIP (mMCPIP) cDNA that included a 596 amino acid open reading frame (GenBank accession number AY920404). The sequence of this cDNA showed 80% identity at the nucleotide level and 82% identity at the amino acid level to that of human MCPIP. The mMCPIP expressed in HEK293 cells strongly cross-reacted with rabbit anti-hMCPIP antibodies.

To verify the data from gene arrays, the inventors examined the production of MCPIP transcripts in human monocytes after treatment with 7 nM MCP-1 by RNA blot analysis with the cloned cDNA for hMCPP as a probe. The results clearly showed that the expected 1.8 kb transcript was found only in MCP-1 treated human monocytes (FIG. 1B).

Since MCPIP contains a putative nuclear localization signal, the inventors tested whether it MCPIP localizes to the nucleus. MCPIP with a C-terminal fusion with EGFP was expressed in HEK293 cells and confocal microscopy was used to examine the localization of the fused MCPIP-EGFP. MCPIP-GFP was found to be localized in the nucleus whereas in the control, GFP was found to be distributed throughout the cell. Propidium iodide that stained the nucleus (red) was co-localized with GFP resulting in the yellow color upon merging of the two images.

Efforts to generate an HEK293 cell line that stably expresses MCPIP-GFP fusion indicated that it caused cell death. In situ TUNEL assay was performed on HEK293 cells after transfection with MCPIP-GFP or GFP alone (control). Transfection with either plasmid resulted in the appearance of robust and equal GFP fluorescence within 16 h after transfection. During the next five days, blebbing of plasma membrane, nuclear condensation and disintegration became clear. After staining, the cells were checked under a fluorescence microscope, the TUNEL positive cells were counted. The results show that expression of MCPIP caused cell death detectable by TUNEL assay. Trypan blue staining also showed that MCPIP expression caused cell death.

The ability of MCPIP to transactivate transcription was tested in an in vitro system. Co-transfection of HEK293 cells with GAL4-MCPIP and the pGal4-Luc reporter demonstrated that MCPIP activated transcription of the luciferase reporter gene showing 865-fold/mg protein after transfection for 24 h, while the positive control containing the well characterized activating transcription factor, ATF4, showed 1263-fold. This result demonstrated that MCPIP could act as a positive regulator of transcription.

To determine whether cell death caused by MCPIP is related to its transcription factor-like activity, the inventors compared the effects of mutations in the putative domains thought to be important for transactivation on the transactivation and the cell death-inducing activities of MCPIP. Mutation of the Zn finger domain that caused a drastic decrease in transactivation also caused drastic reduction in death-inducing ability. Mutation of either proline-rich domain or both caused drastic reduction in cell death-inducing activity whereas mutation of the nuclear localization signal caused a much smaller decrease in cell death, as observed for transactivation. Thus, the structural features that are essential for the transcription factor-like activity are also essential for cell death-inducing activity.

To determine whether expression of MCPIP cause detectable upregulation of genes known to be involved in cell death, microarray analysis was done with RNA isolated soon after MCPIP expression was clearly indicated by the fluorescence of the fused GFP (16 hr) but before cell death was detectable. This gene expression profile showed that MCPIP caused induction of several genes such as, BCL2, CARD (Caspase recruitment domain family), Caspase, CIDE, Death Domain, TNF Receptor, TRAF (TNF Receptor Associated factor) and P53/DNA damage families (Table 1). These gene products are known to be involved in cell death.

Cell death has been associated with development of heart disease and MCP-1 has been implicated to be involved in the development of heart disease. Since MCPIP induces death in cell cultures, the inventors examined whether MCPIP expression is associated with the heart disease in the transgenic mouse model for heart failure, in which cardiac inflammation is induced by cardiomyocyte-targeted expression of MCP-1. Real-time PCR analysis of the MCPIP transcript levels showed that the transgenic animals expressed much higher levels of MCPIP when compared to age and sex-matched wildtype controls. With the development of significant compromise of cardiac function, as measured by echocardiography, MCPIP transcript levels increased dramatically.

In situ hybridization showed that MCPIP transcripts were in cardiomyocytes. In MCP mice MCPIP levels increased as the animals reached 2 months of age and MCPIP staining was associated with the nuclei of cardiomyocytes. When clinical symptoms of heart failure were very obvious at 6 months of age with fractional shortening <20%, MCPIP staining was associated with vacuoles in cardiomyocytes indicating degradation that is a characteristic ultrastructural feature of heart failure in the MCP transgenic mouse as is found also in the human failing myocardium. MCPIP staining was also found in vascular endothelial and smooth muscle cells.

Elevation of CCR2 transcript levels was clear in MCP mice hearts at 2 months of age, prior to the development of clinical manifestation of the heart disease. In situ hybridization clearly showed that CCR2 transcript was present within the cardiomyocytes of MCP mice.

MCPIP expression is associated with ischemic heart failure in the murine model, so the inventors investigated the association between MCPIP expression and human ischemic heart disease. The inventors measured the MCPIP transcript levels in human heart tissue from explanted hearts. Patients were classified as having ischemic cardiomyopathy based on a clinical history of documented coronary artery disease, myocardial infarction, or evidence of ischemia by exercise or pharmacologic stress testing prior to transplantation. In these patients of comparable age, seven were classified as ischemic and the other six non-ischemic. Remarkably, the ischemic hearts showed much higher levels of MCPIP than the non-ischemic hearts.

1. A method for inhibiting the effects of monocyte chemoattractant protein-1 (MCP-1) in a cell, the method comprising adding to the cell an inhibitor of expression, activation, nuclear localization or DNA binding of a monocype chemoattractant protein-1 inducible protein (MCPIP) encoded by a polynucleotide of SEQ. ID. NO: 1.

2. The method of claim 1 wherein the inhibitor of expression of MCPIP is siRNA

3. A method of treating and/or preventing diabetes in a patient in need thereof, said method comprising inhibiting MCPIP in said patient.

4. The method of claim 3, wherein said inhibiting comprises administering to said patient a therapeutically effective amount of a composition comprising an inhibitor of MCPIP and a pharmaceutically acceptable carrier.

5. A method of reducing weight gain and increases of white adipose tissue in a patient at risk comprising inhibiting MCPIP in said patient.

6. The method of claim 5, wherein said inhibiting comprises administering to said patient an therapeutically effective amount of a composition comprising an inhibitor of MCPIP and a pharmaceutically acceptable carrier.


SEQUENCE LISTING (6 sequences)

SEQ ID NO: 1 - DNA, 1800 bp, Homo sapiens
  mRNA (1)..(1800)
  ref <308>: DATABASE ACCESSION NUMBER: GenBank/AY920403
  ref <309>: DATABASE ENTRY DATE: 2005-02-05
  ref <313>: RELEVANT RESIDUES: (1)..(1800)
atgagtggcccctgtggagagaagcctgtcctggaagccagccccaccatgagtctgtgggaatttgaggacagccacagccgtcagggcaccccaaggccgggtcaagagctggccgctgaggaggcctcggccctggaactgcagatgaaggtggacttcttccggaagctgggctattcatccacggagatccacagcgtcctgcagaagctgggcgtccaggcagacaccaacacggtgctgggtgagctggtgaaacacgggacagccaccgagcgggagcgccagacctcaccggacccctgccctcagctccctctagtcccgcggggtggtggcacccctaaggctcccaacctggagcctccactcccagaagaggaaaaggagggcagcgacctgagaccagtggtcatcgatgggagcaacgtggccatgagccatgggaacaaggaggtcttctcctgccggggcatcctgctggcagtgaactggtttctggagcggggccacacagacatcacagtgtttgtgccatcctggaggaaggagcagcctcggcccgacgtgcccatcacagaccagcacatcctgcgggaactggagaagaagaagatcctggtgttcacaccatcacgacgcgtgggtggcaagcgggtggtgtgctatgacgacagattcattgtgaagctggcctacgagtctgacgggatcgtggtttccaacgacacataccgtgacctccaaggcgagcggcaggagtggaagcgcttcatcgaggagcggctgctcatgtactccttcgtcaatgacaagtttatgccccctgatgacccactgggccggcacgggcccagcctggacaacttcctgcgtaagaagccactcactttggagcacaggaagcagccgtgtccctatggaaggaaatgcacctatgggatcaagtgccgattcttccacccagagcggccaagctgcccccagcgctctgtggcagatgagctccgtgccaatgctctcctctcaccccccagagccccaagcaaggacaaaaatggccggcggccttcaccttcatcccagtccagctctctgctaacagagagtgagcagtgcagcctggatgggaagaagctgggggcccaggcatccccagggtcccgccaagagggtctaacacagacctatgccccatcaggcaggagcctcgcacctagcgggggcagtggcagcagctttgggcccacagactggctcccacagacgctggactcactcccgtacgtctcccaggattgcctggactcgggcattggctccctggagagccagatgtcggaactttggggggttcgaggaggaggccctggtgagccgggcccaccccgagccccttacacgggctacagtccctatggatctgagctcccagccaccgcagccttctctgcctttggccgggccatgggtgctggccacttcagtgtccctgccgactacccacccgcgccccctgcctttccacctcgagagtactggtctgaaccatacccactgcccccacccacatcagtccttcaggagcccccagtgcagagcccaggggctggcaggagcccgtggggcagggcaggcagcctggccaaggagcaggccagcgtgtatactaagctgtgtggtgtgtttcccccgcacctggtggaggctgtgatggggcgcttcccacagctcctggacccccagcagctggctgccgagatcctctcctacaagtcccagcaccccagtgagtaa

SEQ ID NO: 2 - protein, 599 aa, Homo sapiens
  PEPTIDE (1)..(599)
  ref <308>: DATABASE ACCESSION NUMBER: GenBank/AY920403
  ref <309>: DATABASE ENTRY DATE: 2005-02-05
  ref <313>: RELEVANT RESIDUES: (1)..(599)
MSGPCGEKPVLEASPTMSLWEFEDSHSRQGTPRPGQELAAEEASALELQMKVDFFRKLGYSSTEIHSVLQKLGVQADTNTVLGELVKHGTATERERQTSPDPCPQLPLVPRGGGTPKAPNLEPPLPEEEKEGSDLRPVVIDGSNVAMSHGNKEVFSCRGILLAVNWFLERGHTDITVFVPSWRKEQPRPDVPITDQHILRELEKKKILVFTPSRRVGGKRVVCYDDRFIVKLAYESDGIVVSNDTYRDLQGERQEWKRFIEERLLMYSFVNDKFMPPDDPLGRHGPSLDNFLRKKPLTLEHRKQPCPYGRKCTYGIKCRFFHPERPSCPQRSVADELRANALLSPPRAPSKDKNGRRPSPSSQSSSLLTESEQCSLDGKKLGAQASPGSRQEGLTQTYAPSGRSLAPSGGSGSSFGPTDWLPQTLDSLPYVSQDCLDSGIGSLESQMSELWGVRGGGPGEPGPPRAPYTGYSPYGSELPATAAFSAFGRAMGAGHFSVPADYPPAPPAFPPREYWSEPYPLPPPTSVLQEPPVQSPGAGRSPWGRAGSLAKEQASVYTKLCGVFPPHLVEAVMGRFPQLLDPQQLAAEILSYKSQHPSE

SEQ ID NO: 3 - DNA, 1791 bp, Mus musculus
  mRNA (1)..(1791)
  ref <308>: DATABASE ACCESSION NUMBER: GenBank/AY920404
  ref <309>: DATABASE ENTRY DATE: 2005-02-05
  ref <313>: RELEVANT RESIDUES: (1)..(1791)
atgagtgacccttgtggaacgaagcctgtccaagaatccaaccccaccatgagtctgtggagtcttgaggacagacacagcagccagggtcgacctcagccagaccaggatcctgtggctaaagaggcccctacttccgagctgcagatgaaggtggactttttccgtaaactggggtactcgtcctctgagatccacagtgtcctgcagaagctgggagtccaagcagacaccaacacggtgctaggggaattggtgaagcatggctcagctactgaacgagaatgccaggccctgacggcccccagcccccagccccctctggtgccccggggtggaagcacccccaagccttccactctagaaccctcactcccagaggaggacagagagggcagcgacctgagacctgtggtcatcgacggaagcaatgtggccatgagccatgggaacaaggaagtcttctcttgccggggcattctgctggctgtgaactggtttctggagcgaggccacacagatattaccgtgtttgtgccatcttggaggaaggaacagcctcgaccagatgtgcctatcacagaccagcacatccttcgggaactagagaaaaagaagatcttggtgttcacgccatccaggcgggtcggcggcaagcgcgtggtgtgctatgatgaccgcttcattgtgaagctggccttcgaatccgacggagtggtggtctccaatgacacgtaccgagacctccaaggcgagaggcaggagtggaaacgcttcatcgaggagcggctgctcatgtactccttcgtcaatgacaagttcatgccccctgacgaccctttaggacggcatgggcctagcctggacaacttccttcgtaagaaaccactgccttctgagcacaggaagcagccatgcccctatgggaagaaatgtacgtatgaaatcaagtgccgatttttccaccctgagcggccaagccgtccccagcgctctgtggccgatgagctccgtgccaacgctctcctctcacctcccaggactccagtcaaggacaaaagtagccagaggccttcccctgcctctcagtccagctctgtgtccctagaggctgaaccaggcagcctggatgggaaaaagctgggtgccaggtcatctccgggtccccaccgagaaggctcaccgcagacctgtgctccagctggcaggagcctccctgttagtgggggcagctttgggcccacagagtggcttgcacacacccaggactcactcccatacacctcccaggagtgccttgattcaggcattggttccctggagagccagatgtcagaattatggggcgtgcgaggaggcagccctggggagtcgggccccactcggggcccctatgcaggttatcacagctatggatccaaggtcccagcagcaccttccttttctccttttagaccagccatgggtgctggccacttcagtgtccccaccgactatgtgcccccgccacccacctacccatccagagagtactggtctgagccgtacccattacccccacccactcctgtccttcaggagccccagagacccagccccggggctggtgggggcccctggggcagggtgggtgacctggccaaagaaagggctggtgtatataccaagctgtgtggtgtcttccccccacacctggtagaagctgtaatgagacgcttcccacagctgctggatccgcagcagctggccgcagagatcctgtcttacaagtcccagcacctcagtgagtaa

SEQ ID NO: 4 - protein, 596 aa, Mus musculus
  PEPTIDE (1)..(596)
  ref <308>: DATABASE ACCESSION NUMBER: GenBank
  ref <309>: DATABASE ENTRY DATE: 2005-02-05
  ref <313>: RELEVANT RESIDUES: (1)..(596)
MSDPCGTKPVQESNPTMSLWSLEDRHSSQGRPQPDQDPVAKEAPTSELQMKVDFFRKLGYSSSEIHSVLQKLGVQADTNTVLGELVKHGSATERECQALTAPSPQPPLVPRGGSTPKPSTLEPSLPEEDREGSDLRPVVIDGSNVAMSHGNKEVFSCRGILLAVNWFLERGHTDITVFVPSWRKEQPRPDVPITDQHILRELEKKKILVFTPSRRVGGKRVVCYDDRFIVKLAFESDGVVVSNDTYRDLQGERQEWKRFIEERLLMYSFVNDKFMPPDDPLGRHGPSLDNFLRKKPLPSEHRKQPCPYGKKCTYEIKCRFFHPERPSRPQRSVADELRANALLSPPRTPVKDKSSQRPSPASQSSSVSLEAEPGSLDGKKLGARSSPGPHREGSPQTCAPAGRSLPVSGGSFGPTEWLAHTQDSLPYTSQECLDSGIGSLESQMSELWGVRGGSPGESGPTRGPYAGYHSYGSKVPAAPSFSPFRPAMGAGHFSVPTDYVPPPPTYPSREYWSEPYPLPPPTPVLQEPQRPSPGAGGGPWGRVGDLAKERAGVYTKLCGVFPPHLVEAVMRRFPQLLDPQQLAAEILSYKSQHLSE

SEQ ID NO: 5 - DNA, 24 bp, Homo sapiens
  primer_bind (1)..(24)
cgcatatgagtggcccctgtggag

SEQ ID NO: 6 - DNA, 27 bp, Homo sapiens
  primer_bind (1)..(27)
cgggatccttactcactggggtgctgg